(19) United States
(12) Patent Application Publication
(10) Pub. No.: US 2023/0069552 A1
(43) Pub. Date: Mar. 2, 2023

(54) BIOREACTOR

(71) Applicant: Sepragen Corporation, Union City, CA (US)

(72) Inventor: Vinit Saxena, Pleasanton, CA (US)

(21) Appl. No.: 17/884,369

(22) Filed: Aug. 9, 2022

Related U.S. Application Data
(60) Provisional application No. 63/231,562, filed on Aug. 10, 2021.

(51) Int. Cl. C12M 1/00; C12M 1/12

(52) U.S. Cl. CPC C12M 23/40; C12M 29/04; C12M 23/34; C12M 25/14

(57) ABSTRACT

The bioreactor can use a number of different culture growing structures for culturing cells including a lattice structure and a support matrix structure. The culture growing structure whether it be a support matrix using wicking and/or a lattice structure may be coated with a thermal responsive polymer. The material of interest growing on the culture growing structure can be removed by changing the temperature that the thermal responsive polymer on the surface of the culture growing structure is exposed to and thus in some cases release the thermal responsive polymer along with the material of interest from the remainder of the culture growing structure.

### RELATED APPLICATIONS

This application claim priority to U.S. Provisional Application No. 63/231,562 filed on Aug. 10, 2021, and which the entire contents of which are incorporated herein by reference.

### FIELD OF THE INVENTION

An embodiment of the invention relates generally to cell culture and more specifically to a novel bioreactor for growing cells for culture and harvesting that can use a support matrix and/or lattice structure and/or combination of both.

### BACKGROUND OF THE INVENTION

Pharmacologically significant biological products for research and therapy are manufactured largely using various cell culture technologies (Chu et al., “Current Opinion in Biotechnology” (2201) 12: 180-187). Monoclonal antibodies, recombinant proteins I peptides including vaccines, produced by such technologies, are currently on the market or in active, phased development world wide. World wide demand for large scale cell culture production, therefore, continues to increase.

Currently, the industry standard method for large scale cell culture is suspension perfusion technology. Prior art devices and methods disclose interrupted exposure to oxygenation by continually and alternately the dipping of cells in out of culture media or moving cells in and out of submersion by a moving belt. These methods and devices compromise between minimizing shear stress on the cells and oxygenation. A few examples of current methods used for cell culture are described briefly below

United States Patent No. 20040058434 issued Mar. 25, 2004 to Philippe Gault, describes a reactor for cell and tissue culture which involves mechanical stimulation of tissues or cells and supply of nutrients by way of a culture medium suitable for structural tissues. An optimum levels of nutrient and oxygen supply necessary for growth of cells or tissues, is achieved by reducing the density of cells and the preparation of implants in a variety of forms, compositions and applications.

U.S. Pat. No. 7,033,823 issued Apr. 25, 2006, to King-Ming titled “Chang Cell-cultivating device” teaches a cell culture method and device where a growth substrate capable of providing a large surface area for cell adhesion. By intermittently and periodically providing sufficient oxygen and nutrients to the cells without causing cell death, it also functions also as an oxygenator, a depth filter and a static mixer to maximize the production of cellular products. The optimum levels of oxygenation and nutrient are regulated by controlling the amount of culture medium that comes into contact with the growth substrate means.

United States Patent Application No. filed Mar. 25, 2005, by Code Kind and Phillipe Gault, titled “Bioreactor For Tissue Cultivated In The Form Of a Thin Layer and Uses Thereof” teaches cell culture methods that grow cells on a thin film held between two plates. This method is specifically designed for tissue implants, but not for the growth of cells by direct exposure to liquid/air interface.

All these methods suffer from major disadvantages in that they have to continuously compromise between sufficient movement of culture media across cell membranes to provide them nutrients for sufficient growth and, at the same time provide sufficient O₂/CO₂ gas exchange rates, without limiting rate of movement of these elements, to minimize shear-stress to the cells. This is a serious dilemma, and currently “dealt with” by reducing cell density to levels that are supported by the limited gas exchange rates. They are not suitable for large scale production as they are not directly scalable.

### SUMMARY OF THE INVENTION

To achieve the foregoing and other objects and in accordance with the purpose of the present invention as embodied and broadly described herein, the design is directed broadly to a high performance bioreactor for cell culture.

The bioreactor can use a number of different culture growing structures for culturing cells including a lattice structure and a support matrix structure.

The text below will discuss various concept applied to the lattice structure or the support matrix structure as an example implementation of the culture growing structures. The inventors understand in most cases, when the text states that the lattice structure uses a technique discussed below that this is being stated as an example implementation as the similar technique can also be applied to the support matrix. The inventors understand in most cases, when the text states that the support matrix uses a technique discussed below that this is being stated as an example implementation as the similar technique can also be applied to the lattice structure.

For example, the culture growing structure whether it be a support matrix using wicking and/or a lattice structure may be coated with a thermal responsive polymer. The material of interest growing on the culture growing structure can be removed by changing the temperature that the thermal responsive polymer on the surface of the culture growing structure is exposed to and thus in some cases release the thermal responsive polymer along with the material of interest from the remainder of the culture growing structure. The culture growing structure may be made of cellulose and coated with, for example, a thermal responsive polymer. The culture growing structure may specifically use a support matrix using wicking and having a thermopolymer surface coating.

The thermopolymers can include, for example, poly(N-isopropylacrylamide) p(NIPAm), poly-(ethylpyrrolidone methacrylate) (pEPM), poly[2-(dimethylamino)ethyl methacrylate] (pDMAEMA), hydroxypropylcellulose, poly(vinylcaprolactame), polyvinyl methyl ether, and other similar thermopolymers. Some may attach better to the surface of the matrix whether it be a support matrix or lattice structure, some may release better and or in a more desired temperature range, etc. Many may use pretreatments on the matrix material to make that material more readily work better with the surface coating.

Other surface coatings can include functional groups, denatured protein-based fibers, plasma treatment, sodium hydroxide, combinations of these, etc.

The design provides a high performance bioreactor device and method for culturing biological cells on a matrix such as a support matrix and/or lattice structure. The bioreactor device uses support matrices comprising a porous material having continuous open pores that permit the substantially free transport of liquids and gases through the support matrix. The bioreactor device uses a gravity-assisted capillary or wicking process to evenly distribute a thin layer or film of the nutrient rich, culture medium, across the surface of the porous support matrix where the cells of interest are immobilized. The device also provides for the simultaneous oxygenation of the cells by flowing air across the surface of and through the porous support matrix.

In one embodiment, biological cells are immobilized on the surface of the support matrix with a thin film of culture medium continuously flowing over its surface. In another embodiment, the support matrix is formed of interlacing and interconnected fibers of a material compatible with the biological cells being cultured. Another important feature of the design is the regulation of the flow of culture medium over the support matrix ensuring the maintenance of a thin film of medium over substantially the entire surface of the support matrix. In yet another aspect, such regulation is accomplished by monitoring the back pressure of the air or other gas or gases, such as O₂ or CO₂, introduced into the culture chamber.

The bioreactor device can include the following elements: (a) a culture chamber having an inlet, an outlet, and an interior; (b) a support matrix with a top end and a bottom end, mounted in the interior of the culture chamber for holding biological cells on the support matrix, the support matrix comprising a porous material having continuous open pores, such material being formed of interlacing and interconnected fibers and having a non toxic surface suitable as a substrate for biological cells; the continuous open pores of such material permitting substantially equivalent communication with the interior of the culture chamber from any location on the surface of the support matrix; (c) a first reservoir mounted outside the bioreactor for holding a culture medium; (d) a second reservoir for the culture medium supported at the top end of the reservoir directly above the top end of the support matrix; (e) fluid circulation means having a fluid delivery rate for non-turbulently delivering culture medium to the support matrix, such that the culture medium flows in a thin film over substantially the entire surface of the support matrix to the bottom end of the support matrix and the chamber which is then removed or recycled through the outlet; and (f) circulation means for supplying oxygen or other gas or gases, as are suitable for growth of the cells, to the surface of the support matrix and through the support matrix to the interior of the culture chamber.

In another embodiment, the support matrix divides the interior of the culture chamber into a first region and at least one second region. The device further comprises a gas inlet in communication with the first region and a gas outlet in communication with the at least one second region, the gas inlet and gas outlet being operationally connected to a regulated source of air for the culture chamber that provides a flow of air from the gas inlet through the support matrix to the gas outlet. Preferably, the first region of the culture chamber has a first pressure and the at least one second region of the culture chamber has a second pressure, such that the first pressure is substantially equivalent to the second pressure, i.e. there is no back pressure due to the culture medium impeding the flow of air through the support matrix.

In another aspect, the bioreactor device further comprises a regeneration means operationally associated with the fluid circulation means, the regeneration means (a) receiving the culture medium from the outlet, (b) optionally removing waste material or extracting product from such culture medium, (c) optionally replenishing nutrients to such culture medium, and (d) delivering the culture medium to the fluid circulation means.

In yet another embodiment, a method for high performance cell culture comprises the following steps: (a) providing a culture chamber having an inlet, an outlet, an interior, and a support matrix mounted in the interior for holding biological cells, the support matrix comprising a porous material having continuous open pores, such material being formed of interlacing and interconnected fibers or porous foam, and having a non toxic surface suitable as a substrate for biological cells, the continuous open pores of such material permitting substantially equivalent communication with the interior of the culture chamber from any location on the surface of the support matrix; (b) introducing a culture medium containing biological cells into the culture chamber and allowing the biological cells to become immobilized on the support matrix; and (c) non-turbulently delivering a flow of culture medium to the support matrix, such that (1) the flow of culture medium travels in a thin film over substantially the entire surface of the support matrix to a reservoir at the bottom end of the support matrix and through the outlet, (2) substantially none of the continuous open pores of the support matrix are flooded by the flow of culture medium, (3) circulating fluid reaches the bottom of the support matrix, turbulent free, thus preventing foam formation, and (4) simultaneously flowing a stream of air gently across and through the surface of the support matrix such that there is no back pressure generated by the flow of the medium across the surface of the matrix.

The design can include a bioreactor device and method available that provides maximal oxygen transfer to all cells in the culture in a substantially equivalent manner while, at the same time, supplies sufficient nutrients for cell growth in high density, convenient product harvesting and ready scalability.

The design can provide a high performance and high density bioreactor for cell growth and culture.

The design can provide a method and device for cell culture where the cells are continuously, rather than intermittently, bathed in a culture medium with zero shear-stress, while simultaneously and continuously supplied essential nutrients and exposed to optimal O₂/CO₂ gas exchange.

The design can provide a novel bioreactor for the culture of cells without having to immerse the cells in the growth medium.

The design can provide a bioreactor for cell culture wherein the cells are continuously and simultaneously fed and aerated to achieve maximum growth in a relatively short time.

The design can provide a bioreactor which is directly scalable to workable proportions.

The design can include an “accelerated wicking” process for the distribution of the growth medium across the surface of cell support matrix.

Additional objects, advantages and novel features of the design will be set forth in part in the description and drawings which follow, and in part will become apparent to those skilled in the art upon examination of the following or may be learned by practice of the design. The objects and advantages of the design may be realized and attained by means of the instrumentalities and combinations particularly pointed out in the appended claims.

### BRIEF DESCRIPTION OF THE DRAWINGS

FIG. 1A is a schematic of a bioreactor assembly for cell culturing with a lattice structure as presently disclosed, according to embodiments of the design.

FIG. 1B is a photograph of a bioreactor assembly for cell culturing with a lattice structure as presently disclosed, according to embodiments of the design.

FIG. 1C is a schematic showing a front-on view of a bioreactor assembly with a lattice structure as presently disclosed with the lattice structure depicted to be suspended out of media and fluid is recirculated through the bioreactor system, according to embodiments of the design.

FIG. 2 shows a schematic (left) of a lattice structure according to some embodiments of the design where the lattice structure has a first channel pore surface and a second channel pore surface. A photograph (right) of a lattice structure with the plurality of channel pores, core passage, and sampling ledge members depicted, according to some embodiments of the design.

FIG. 3A is a schematic showing sequential (left to right) layer three-dimensional (3D) printing of the lattice structure 10, according to embodiments of the design.

FIG. 3B is a schematic depicting one embodiment of forming the channels and channel pores 15 of the lattice structure 10, according to embodiments of the design.

FIGS. 4A, 4B, 4C, 4D, 4E, and 4F are each scanning electron microscope (SEM) images of one or a few channel pores in a polylactic acid (PLA) 3D printed lattice structure where FIG. 4A is a view of an exterior side of the lattice structure 10. FIG. 4B is a view of an interior section of the lattice structure 10. FIG. 4C is a top-down view of the lattice structure 10 with the channel pores 15. FIG. 4D is a zoomed in image of the exterior side of FIG. 4A of the lattice structure 10. FIG. 4E is a zoomed in image of the interior section of FIG. 4B of the lattice structure 10, and FIG. 4F is a zoomed in top-down view of FIG. 4C of the lattice structure 10. The white squares denote a magnified view of that area seen in the second row below the top row.

FIG. 5 shows the results from Computational Fluid Dynamic (CFD) modeling on a modeled lattice structure where the upper model is a velocity contour showing the calculated results of fluid velocity in the lattice structure and the lower model shows the calculated results of shear stress in the lattice structure, according to embodiments of the design.

FIG. 6 is a graph 600 of inlet flow rate versus average wall shear where the lattice structure was modeled in ANSYS and tested at various flow rates using Fluent. The strain rate was converted into shear stress using Equation 1, according to embodiments of the design.

FIG. 7 shows human mesenchymal stem cells (hMSCs) 700 imaged on a PLA lattice structure used in a bioreactor.

FIGS. 8A-8D are data graphs 800 from a hMSC bioreactor culture of varying oxygen tension compared to static hMSC culture grown under the same oxygen amounts and harvested on day 7.

FIG. 9 is an exemplary graph of data 900 obtained from flow cytometry of hMSC grown on a lattice structure in a bioreactor system according to embodiments of the design in which hMSC cells were harvested day 7 from varying oxygen tension.

FIG. 10A is an exemplary graph 1000 of hMSC biomarker characterization using flow cytometry.

FIG. 10b shows exemplary data of an overlaid flow cytometry image 1010 of 1.5% O2 primed hMSC cultures from day 7 in dynamic bioreactors (with lattice structure) showing positive markers and negative markers, according to embodiments of the design.

FIG. 10C shows exemplary data of an overlaid flow cytometry image 1020 of 1.5% O2 primed normoxic polystyrene static control culture showing positive markers and negative markers, according to embodiments of the design.

FIG. 11 shows exemplary light microscope images 1100 of stem cell induction in which cells were harvested day 7 from bioreactor (with lattice structure) and cultured in respective differentiation media following specialized protocols.

FIGS. 12A and 12B show exemplary bar graph 1200 data comparing culture methods including dynamic bioreactor (with PLA lattice structure), spinner flasks, and static growth in t75 flasks as indicated, in which FIG. 12A presents doubling time and FIG. 12B presents the specific growth rates of the corresponding hMSCs.

FIG. 13 is a side view illustrating a cell culture bioreactor.

FIGS. 14A and 14B are side views illustrating culture medium flow over a support matrix of the cell culture bioreactor, versus under a condition wherein support matrix pores are flooded;

FIG. 15 is a perspective view illustrating a bioreactor system employing a culture chamber;

FIG. 16 is a perspective view illustrating another embodiment of a cell culture bioreactor;

FIG. 17 is another perspective view illustrating the cell culture bioreactor of FIG. 16; and

FIG. 18 is a sectional view illustrating the cell culture bioreactor of FIG. 16.

### DETAILED DESCRIPTION OF THE INVENTION

The design provides a method, system, and device for supporting large-scale continuous, or batch culturing of biological cells, by culturing cells directly on the support matrix. In an alternative mode, the design also provides a method and device for continuously aerating, and in particular oxygenating a culture medium used to support a bioreactor by acting as a gas exchanger (a “lung”) only, with no cells immobilized on the support matrix. In this aspect cells are NOT grown directly on the “lung”, but are grown, in a conventional bioreactor (i.e. hollow fiber or suspension culture), partitioned from the support matrix. The support matrix oxygenates the culture fluid, but cells are grown in a connected (but partitioned) conventional bioreactor.

The design provides the simultaneous and continuous oxygenation and nourishment of the biological cells being cultured. This is achieved by the use of a support matrix that is a porous material having continuous open pores for simultaneously aerating biological cells and at the same time supplying nutrients for their growth and sustenance. The material for the support matrix is formed of interlacing and interconnected fibers, or porous foam, that are non toxic to cells but with a surface suitable for a culture medium to flow over it in a thin layer or film, such that even the continuous flow of a thin layer of culture medium, air flow through the material will be substantially unrestricted. Typically, a thin layer or film of the growth or culture medium has a thickness of from a few μm, e.g. 1 μm, to about 1 mm, i.e. 100 μm. As used herein, the term “thickness” in reference to a thin film or layer of culture medium means the perpendicular distance from the surface of the support matrix to the liquid-air interface of the culture medium. “Substantially unrestricted” in reference to air flow through a support matrix can mean that the presence of a flow of the culture medium over the surface of such support matrix does not impede the passage of air through the pore system of the support matrix. Such substantially unrestricted flow of air or any other gas conducive to the growth of the cells of interest, through a support matrix is important to ensure that every location on the support matrix is substantially equivalent with respect to the exchange of air or gases between the culture medium and the ambient atmosphere. The unimpeded flow of gas through a support matrix in an embodiment of the device depends on several factors including, but not limited to, the flow rate of the culture medium through the support matrix, the viscosity of the culture medium, the surface tension forces between the culture medium and the support matrix surface, the flow rate of the air, the detailed structure of the support matrix, and the like.

The bioreactor comprises a three dimensional culture chamber, cylindrical, rectangular or any other shape capable of easy handling. It may be constructed of glass, or any other chemically non-reactive, bio-compatible material like ceramic, stainless steel and the like. A support matrix comprising a three-dimensional porous sponge or reticulated foam or wicking filter or other materials used in humidifiers, is mounted on top of the chamber directly above the support matrix. A nonturbulent recirculation/distribution system is a second reservoir for the culture medium is mounted directly above the support matrix. A first reservoir for holding the culture medium is mounted either at the base of the culture chamber and/or connected to an exterior reservoir. A second reservoir for holding the culture medium is mounted at the top of the support matrix. The culture medium from the first reservoir is pumped into the second reservoir through tubing whose delivery end is submerged in the liquid contained in the second reservoir to eliminate surface splashing turbulence. The second reservoir is designed to allow even distribution of over-flow which then flows directly onto the top of the support or wicking matrix, again avoiding air/fluid splashing turbulence. This turbulence free medium distributor allows very rapid, non-turbulent delivery of culture medium to the top of the wicking matrix.

The fluid delivered to the top of the support or wicking matrix flows evenly and uniformly down the matrix by gravity-assisted capillary/wicking flow and collects at the bottom of the chamber. An outlet for removal or recycling of the spent medium is provided near the bottom of the culture chamber. The spent medium is then pumped back to the first reservoir for recycling or is discarded. The bottom or lower end of the matrix is positioned to just touch or slightly submerge in the spent culture medium flowing down the support matrix. This turbulence free circulation of the medium through the entire system, substantially eliminates undesirable and denaturing foaming and other effects.

Support matrices suitable for use in the device may be synthetic or natural porous, three-dimensional matrices. Pore sizes may vary from ten p microns to 100 or more to allow the capture, entrapment and/or binding of the cultured cells up to ten or more millimeters to allow free, unobstructed gas exchange throughout the matrix even while the culture medium is flowing through it. Such materials must also be capable of sterilization and stable over a long period of use. They must also be chemically modifiable for certain types of cell growth. They must all be stable over a long period of use. Pore size of the matrix must be large enough to support high density cell growth and still allow free flow of air throughout the matrix. The support matrix chosen must be such that the flow of fluid across its surface is achieved by any or a combination of gravity assisted capillary/wicking and gravitational forces as defined herein, rather than by direct pumping pressure required in gas permeable membrane systems, which generates fluid turbulence. Materials for the support matrix must also have adequate capillary and adsorption characteristics (“wicking) to allow a rapid, thin film of fluid to traverse the fibrous structures of the matrix. Suitable materials include but are not limited to natural vegetable sponge, more specifically “loopha” sponge, or animal sponges. Synthetic sponges made from polyurethane or other synthetic materials which meet the above criteria may be utilized. Other hydrophilic, hydrophobic charged or neutral matrices are also suitable for use as the support matrix, depending on the nature and properties of the cells of interest. Preferred materials include celluose based expanded “Wicking Filter” such as those used in humidifiers to maximize air/wicking liquid surface areas, macroporous poly (DL-lactide) foams, loofa sponge, three-dimensional polyvinyl-alcohol matrices and the like.

The growth or culture medium is distributed across and through the support matrix in a continuous, rapidly flowing, thin fluid film can bathe the cultured cells (growing in the pathway of the of the fluid flow) thereby maintaining an open pathway, throughout the support matrix to allow for a continuous gas exchange. The three-dimensional porous structures present a large liquid/air surface and minimize saturation or flooding. Examples of cell types for culture and harvesting, using the bioreactor include but are not limited to monoclonal antibody secreting hybridoma cells derived from mice, rats, rabbit or human, Eukaryotic cells, biochemical markers, recombinant peptides or nucleotide sequences of interest, proteins, yeast, insect cells, stable or viral infected, avian cells or mammalian cells such as CHO cells, monkey cells, lytic products and the like for medical, research or commercial purposes.

Culture media normally used for tissue culture are suitable for use as a culture medium in the bioreactor. Examples include but are not limited to DMEM or RPMI formulations known in the art, with or without fetal bovine serum, penicillin, L-glutamine, streptomycin and other culture additives in common use. Other nutrients used for specific situations and which promote the growth of particular cells of interest may also be incorporated into the culture medium.

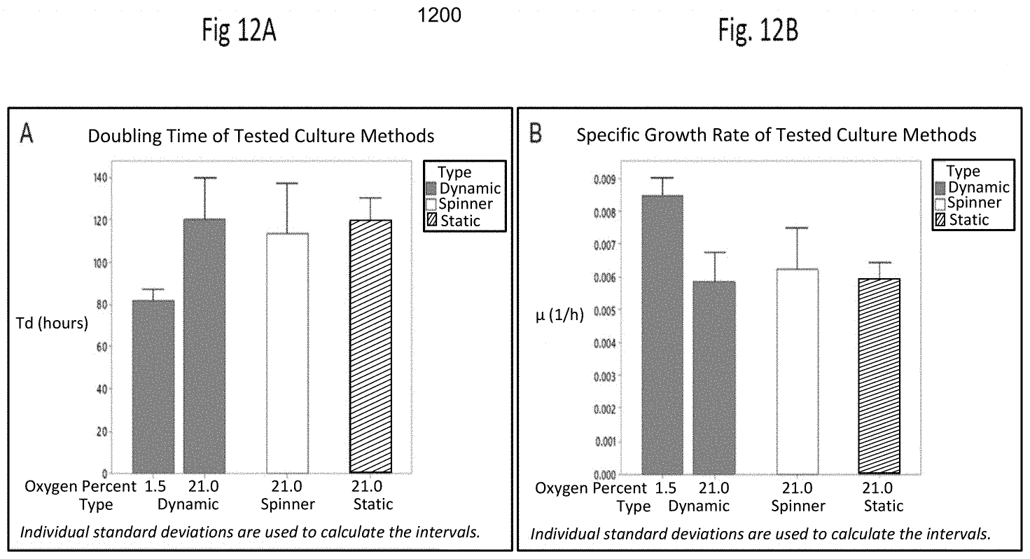

A typical embodiment of the device is illustrated in FIG. 13. The device 100 comprises (i) a culture chamber 102 with a lid 114, (ii) a support matrix 104 in the form of a hollow cylinder having an interior 150, a top end 110, a bottom end 108, and being mounted inside the chamber 102 with the top end 110 sealably attached to a manifold 116 so that whenever reservoir 106 of culture medium is present, the interior 150 communicates with the region 152 only through pores of the support matrix 104, (iii) an inlet 130 for introducing culture medium and/or biological cells to the support matrix 104, (iv) an outlet 122 for removing culture medium from the chamber 102 for regeneration or removal of waste products and/or desired products, (v) fluid circulation means 124 for driving the culture medium through the culture chamber 102, and (vi) air or gas conditioning means 126 for driving air or other gases into the chamber 102 for circulation through support matrix 104. Culture medium from the inlet 130 is delivered to manifold 116 that non-turbulently distributes culture medium to the top end 110 of the support matrix 104.

The uniform non-turbulent distribution of the culture medium to the top end 110 of the support matrix 104 can be accomplished in many different ways and is a matter of design choice of one of ordinary skill in the art. As illustrated in FIG. 13, manifold 116 is a receptacle that receives the culture medium 112 which then flows through multiple ports 118 spaced around the manifold 116 so that culture medium flows 134 through such ports onto the top surface of the support matrix 104. A number of ports are selected so that the flow of the culture medium is evenly, or uniformly, distributed to the top surface of the support matrix 104. Receptacle 112 optionally may not be provided with ports 118 for flow of culture medium but by over flow of the culture medium after it fills the receptacle. The culture medium then flows, for example by gravity, and capillary forces, from the top end 110 of the support matrix 104, through the support matrix 104 as illustrated by arrows 138, to the bottom end 108 of the support matrix 104, and into the reservoir 106. This non-turbulent flow prevents the formation of foam, a serious problem seen with many conventional systems.

From the reservoir 106, the culture medium is then driven, or siphoned, out of the chamber 102 through the outlet 122. The support matrix 104 extends into the reservoir 106 only enough to maintain a fluid connection between the bottom surface of support matrix 104, preventing foam causing turbulence, and reservoir 106; thus, distance 140 is close to zero, preferably, at most 1-2 mm.

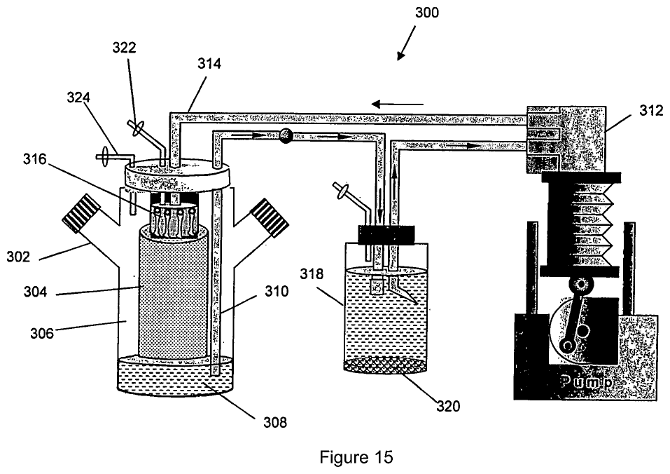

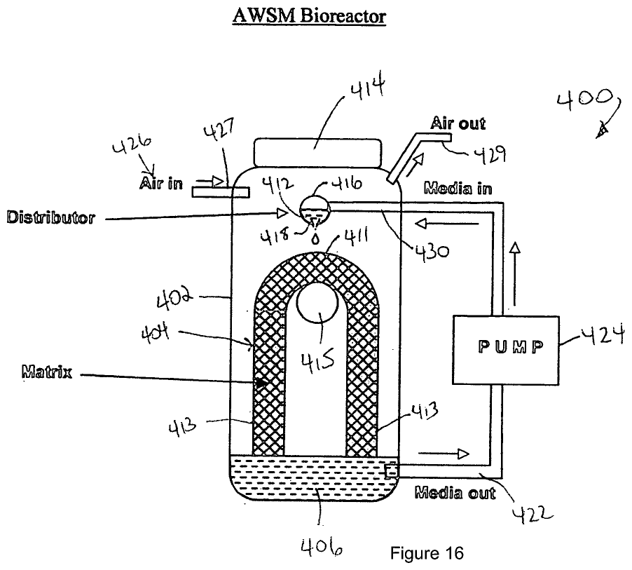

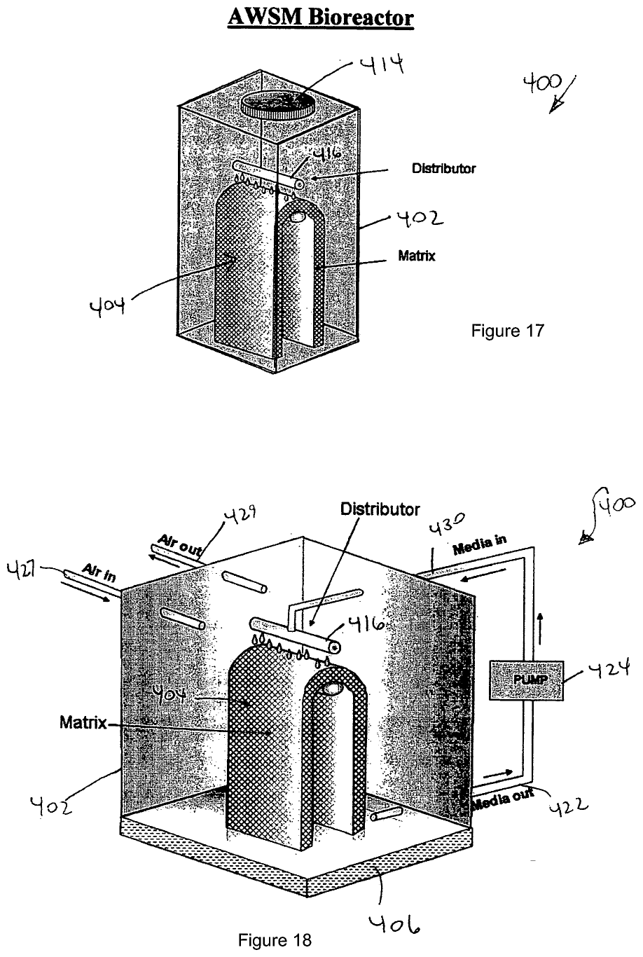

In one aspect, a conditioned atmosphere optimized for the objectives of the culture (e.g. growth rate, product synthesis, etc.) is flowed into the first interior region 150 of the culture chamber 102 through a gas inlet 128. Preferably, pressure sensor 120 is operationally connected to the gas inlet 128 so that any back pressure or resistance to a steady gas flow can be detected. From the first interior region 150, atmosphere flows 136 through the pores of the support matrix 104 into the second interior region 152 of the culture chamber 102, after which it is removed via an exhaust port 132, which may be a simple, sterile filtered vent, or other conventional means to maintain a conventional sterile exhaust system, preventing microbial contamination from back-flowing into the bioreactor sterile field, may carry the atmosphere to an atmosphere conditioning station where it is prepared for recycling. Another embodiment of the Device 400 is illustrated in FIGS. 16-18. Device 400 comprises (i) a culture chamber 402 with a lid 414, (ii) a support matrix 404 in the form of a sheet having a middle portion 411, ends 413, and being mounted inside the chamber 402 with the middle portion 411 draped over a support rod 415, (iii) a manifold 416 positioned above the middle portion 411 and the support rod 415 so that whenever reservoir 406 of culture medium is present, (iv) inlet 430 for introducing culture medium and/or biological cells through the manifold 416 to the support matrix 404, (v) outlet 422 for removing culture medium from the chamber 402 for regeneration or removal of waste products and/or desired products, (vi) fluid circulation means 424 for driving the culture medium through the culture chamber 402, and (vii) atmosphere conditioning means 426 having an air inlet 427 and an air outlet 429 for driving atmosphere into the chamber 402 for circulation through the support matrix 104. The culture medium from the inlet 430 is delivered to the manifold 416 that non-turbulently distributes culture medium to the middle portion 411 of the support matrix 404.

The uniform non-turbulent distribution of culture medium to the middle portion 411 of the support matrix 404 can be accomplished in many different ways and is a matter of design choice of one of ordinary skill in the art. As illustrated in FIGS. 16-18, the manifold 416 is a receptacle that receives culture medium 412 which then flows through multiple ports 418 spaced along the manifold 416 so that culture medium flows through such ports onto the middle portion 411 of the support matrix 404. A number of ports are provided so that the flow of culture medium is evenly, or uniformly, distributed to the middle portion 411 of the support matrix 404. The culture medium then flows, for example by gravity, and capillary forces, from the middle portion 411 of the support matrix 404, through the support matrix 404 as illustrated by the arrows 438, to the ends 413 of the support matrix 404, and into the reservoir 406. This non-turbulent flow prevents the formation of foam, a serious problem seen with many conventional systems.

From the reservoir 406, the culture medium is then driven, or siphoned, out of the chamber 402 through the outlet 422. The ends 413 of the support matrix 404 extend into the reservoir 406 only enough to maintain a fluid connection between the bottom surface of the support matrix 404, preventing foam causing turbulence, and the reservoir 406.

With this embodiment, similar to the first embodiment, a conditioned atmosphere optimized for the objectives of the culture (e.g. growth rate, product synthesis, etc.) is flowed into the culture chamber 402 through the air inlet 427. Preferably, a pressure sensor (not shown) is operationally connected to the air inlet 427 so that any back pressure or resistance to a steady gas flow can be detected. The atmosphere flows through the pores of the support matrix 404 after which it is removed via the exhaust port 429, which may be a simple, sterile filtered vent, or other conventional means to maintain a conventional sterile exhaust system, preventing microbial contamination from back-flowing into the bioreactor sterile field, may carry the atmosphere to an atmosphere conditioning station where it is prepared for recycling.

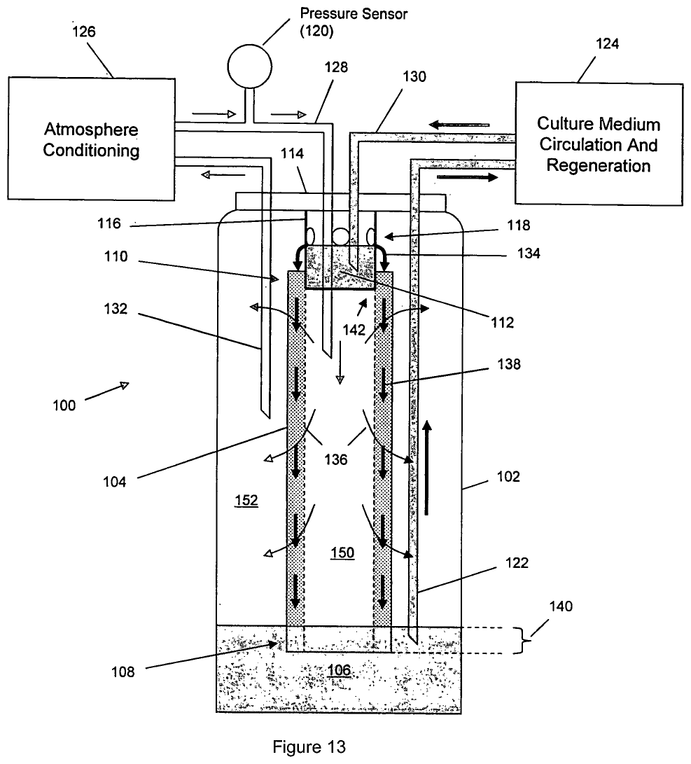

FIG. 14A illustrates the structure of a support matrix material and how it interacts with a flow of culture medium in the device. The structure 206 is a blow-up of small section 202 of the support matrix 204 disposed in the culture chamber 200. As mentioned above, in one aspect, the support matrix 204 comprises a porous material having continuous open pores 210 that are formed from interlacing and interconnected fiber, or porous foam, which are illustrated in blow-up 206. Under desired operation, culture medium 211 flows 208 in a thin film over the surface of the fiber 212. A flow rate is selected so that the pores 210 are un-obstructed so that gas can freely flow 209 through the support matrix 204. As illustrated in FIG. 14B, when a flow rate for the culture medium is too high 216, a volume of the culture medium flows through 218, and thereby floods 220, multiple pores. Such flooding is undesirable as it interferes with the free access of local regions of support matrix 204 to air circulation. It may be noted here that in the currently used submerged sponge systems all of the pores are flooded as marked by X and X which leads to a significantly less efficient gas, and nutrient exchange.

The following experiments demonstrate the efficiency of the present device over prior art methods for cell culture.

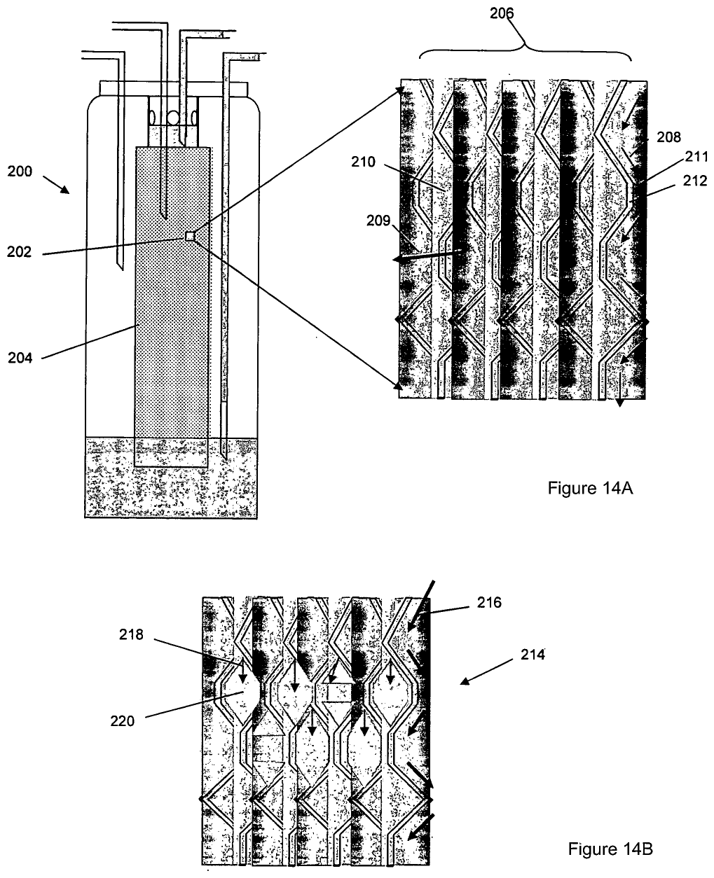

In the first experiment, a one liter Bellco flask with 2 side arms was assembled as illustrated in FIGS. 13-15. The sterilized bioreactor was populated with cells as follows:

Five “T”-75 flasks containing a culture medium consisting of DMEM (Delbecco's Modified Eagle Media)+10% FBS (fetal bovine serum)+4 mM fresh glutamine were inoculated with a hybridoma cell line producing, monoclonal antibody and incubated in a 6% CO₂ 37° C. incubator until the cell count reached a concentration of 10⁵ viable cells/m L. The cells were then transferred into the washed and sterilized bioreactor. The bioreactor was incubated at 37° C. The medium circulation rate was set to about 35 ml/min. The air pump, which circulated the air with 6% CO₂, was adjusted to flow at approximately 3 bubbles per second. For the first 10 days 250 mL of the culture medium was exchanged daily. On day-11 the harvesting of the material of interest began.

In experiment two, two “T”-75 flasks were set up to allow growing to a confluence of approximately 5×10⁵ cells/mL. The cells were then allowed to grow to the “death phase” which took about 3 weeks. The spent medium was frozen for later analysis and were used as control. Initially, 250 mls. Samples of spent medium was collected as harvest and frozen. Then 250 ml of fresh medium was used to replace the spent medium. On week four, the replacement volume was reduced to 50 mls/day. Cell viability was monitored daily by microscopic examination. Cell viability stabilized at approximately 80% throughout the remaining time. After six weeks the culturing was terminated as planned.

Results indicated that cells grew much more rapidly and to a higher cell density and thus, the end product, using the device and method of the present design compared to prior at batch processing and submersion methods.

The present design thus presents a novel device and method for efficient gas exchange supporting cell culture systems which provides a rapid continuous flowing thin film (and thus, high surface area) of nutrient containing, gassed, culture medium. Foam causing turbulence during rapid recycling of the culture medium is avoided by a novel recycling overflow/reservoir system. Such a system places recycling medium gently onto the porous support matrix. Internal leaking is avoided by the design of the system, allowing any overflow (i.e., when the recycling rate is greater than the absorbency of the support matrix.) to be kept within the recycling aseptic reservoir environment.

In addition, the present design uses less pressure (thus less energy, less system stress, less complexity) than current gas permeable membrane methods (such as hollow fiber or spiral wound gas exchangers). Pressure driven gas exchange is avoided by substituting gravity and capillary forces to provide the large surface-area gas exchange process.

Furthermore, the present design allows significantly more efficient gas exchange than mixing, shaking, rocking, or sparging, because of the significantly increased surface area. The device is directly or linearly scalable such that gas exchange diffusion rates are maintained when scaling up from small units to large units. The scaling up is accomplished by maintaining the thickness and height of the support matrix and the corresponding size of the culture chamber, but expanding the width to a useful production size. Linear scalability reduces manufacturing development time, significantly reducing development costs and time-to-market.

A lattice structure for culturing cells in a bioreactor is effective for culturing high density cells and maintaining cell type homogeneity. The lattice structure includes a plurality of channels forming a set of channels, each of the plurality of channels extending between a first channel pore surface and a second channel pore surface and each of the plurality of channels having a first channel pore and a second channel pore altogether forming a plurality of channel pores on each of the first channel pore surface and the second channel pore surface, wherein each of the channel pores has an area of between about 0.01 mm2 to about 1 mm2, and wherein the lattice structure is made of a biocompatible rigid material having a Young's modulus value of at least 0.5 GPa.

Next, a more detailed discussion of the lattice structure for culturing cells is discussed; and most especially, a lattice structure for culturing adherent cells in a bioreactor.

A robust method for scalable bioreactor cell culturing is ideally designed to render high density and often times high purity cell yields. In most conventional bioreactors, cells grow in the liquid nutrient medium which also serves as the means by which dissolved oxygen is transported to the cell surface. Typically, air (e.g., oxygen (O2) and carbon dioxide (CO2)) is dissolved in the medium by bubbling gas into it followed by stirring or rocking of the medium to aid in its diffusion to the cells. The oxygen which is known to have poor solubility in culture medium is rapidly depleted as the cell density rises. Oxygen depletion and poor mixing results in apoptosis of the cells, and an eventual “crash” or death of the culture. To prevent rapid depletion of oxygen in the culture, the bubbling, stirring, and/or rocking rates are all increased over time to thereby increase oxygen delivery. Unfortunately, these actions cause an increase in shear rates which fragile (e.g., mammalian) cells cannot handle. In perfusion systems, rates of media exchange are often increased to enhance oxygen transport. However, this renders an increase in media consumption, and increased cost of goods.

To reduce shear stress on more sensitive cells, cellulose-based matrices in a bioreactor system using gravity and capillary action have been used in which the cells are captured in the pores of the cellulose-based matrix. While cellulose-based matrices provide a low shear environment, for the culturing of adherent cells such as human mesenchymal stem cells (hMSCs), cellulose-based matrices are not effective for promoting adhesion of these anchorage-dependent cells some of which may also require high purity—e.g., maintaining potency stemness.

Thus, there remains a need for a robust method of culturing cells in a bioreactor that provides a low shear stress environment and yields high density and high purity/homogeneity of cells.

The design provides an apparatus, systems, and methods in which a lattice structure has repeating channels throughout to allow for non-turbulent media flow through the lattice, while providing an effective amount of surface area for robust growth of adherent cells.

Aspects of the contemplated lattice structure for culturing cells include a plurality of channels forming a set of channels, each of the plurality of channels extending between a first channel pore surface and a second channel pore surface and each of the plurality of channels having a first channel pore and a second channel pore altogether forming a plurality of channel pores on each of the first channel pore surface and the second channel pore surface. Each of the channel pores has an area of between about 0.01 mm² to about 1 mm², and the lattice structure is made of a biocompatible rigid material having a Young's modulus value of at least 0.5 GPa.

In some embodiments, the lattice structure includes plurality of channels that are approximately uniform with no more than 2% variation between any two of the plurality of channels. Preferably, the first channel pore surface and the second channel pore surface have a diameter or diagonal of between about 5 mm to 100 mm. It is further contemplated that that the lattice structure has a total space-occupying volume of between about 2 cm³ to about 800 cm³.

Some embodiments include a lattice structure in which each of the channel pores is approximately the same size having no more than 2% variation in any dimension, and each of the channel pores has a diameter or diagonal of between about 0.1 mm to 1 mm. In similar embodiments, each of the channel pores of the lattice structure has approximately the same area size and the lattice structure further comprises a spacing distance between all of the channel pores that is equal to the area size of the channel pores.

In preferred embodiments, the lattice structure is made from a biocompatible rigid material having a Young's modulus value of at least 1.0 GPa. Exemplary biocompatible rigid materials include polylactic acid (PLA), polystyrene, polycarbonate (PC), polystyrene (PS), polyethylene terephthalate glycol (PETG), thermoset polyurethane (TPU), polycaprolactone (PCL), or acrylonitrile butadiene (ABS).

In some embodiments, the first channel pore surface and the second channel pore surface of the lattice structure has a perimeter edge forming a circular, oblong, square, octagonal, hexagonal, or rectangular shape. Additionally or alternatively, each of the plurality of the channel pores have a square or circular shape.

In additional embodiments, the lattice structure includes a hollow passage extending from the first channel pore surface to the second channel pore surface, the hollow passage allowing for insertion of a member for securing the lattice structure. In other additional embodiments, a removable substrate made of the biocompatible material and removably attached to at least one of the first channel pore surface or the second channel pore surface.

In more preferred embodiments, the lattice structure includes the first channel pore surface and the second channel pore surface each having a diameter or diagonal of between about 10 mm and 60 mm and each of the plurality of channel pores is of between about 0.2 mm and 0.4 mm, the lattice structure further comprising a spacing distance between each of the plurality of channel pores equal to the diameter or diagonal of the pores.

Additionally or alternatively, the lattice structure may have a surface coating. In particular embodiments, the surface coating includes a functional group capable of binding to the surface of a cell to be cultured. Additional surface coatings include denatured protein-based fibers, thermoresponsive polymers (i.e., thermopolymers), plasma treatment, and/or sodium hydroxide. Examples of denatured protein-based fibers include gelatin, collagen type 1, collagen type 2, fibronectin, and/or laminin. Examples of thermoresponsive polymers include poly(N-isopropylacrylamide) p(NIPAm), poly-(ethylpyrrolidone methacrylate) (pEPM), poly[2-(dimethylamino)ethyl methacrylate] (pDMAEMA), hydroxypropylcellulose, poly(vinylcaprolactame), and/or polyvinyl methyl ether.

A system for culturing cells is contemplated, wherein the system includes a bioreactor assembly including the presently disclosed lattice structure. In particular, the lattice structure may be used in/incorporated into a perfusion bioreactor for cell expansion and recovery. In some embodiments, the volume ratio of the lattice structure to the volume of the bioreactor is about 1:6 to about 1:8.

Additional aspects of the present disclosure include methods for maintaining cell type homogeneity. These methods include culturing a single cell type using the presently disclosed lattice structure at flow rates of about 0.25 ml/min to 0.5 ml/min. In preferred embodiments, methods of maintaining cell type homogeneity produce cell cultures having at least 95% cell type homogeneity.

Using the disclosed lattice structure incorporated into a bioreactor system allows for facile methods of seeding cells by gravitational or perfusion flow. In further embodiments, the culturing of cells on the lattice structure in a bioreactor system may be carried out under normal and hypoxic oxygen conditions. For example, the oxygen (O2) levels may vary from 1% up to 20%.

Again the Figures for this section are as follows.

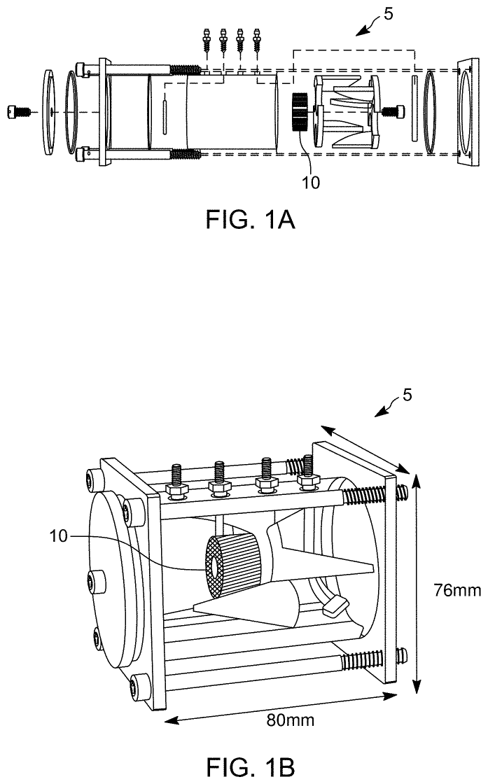

FIG. 1A is a schematic of a bioreactor assembly 5 for cell culturing with a lattice structure 10 as presently disclosed, according to embodiments of the design.

FIG. 1B is a photograph of a bioreactor assembly 5 for cell culturing with a lattice structure 10 as presently disclosed, according to embodiments of the design.

FIG. 1C is a schematic showing a front-on view of a bioreactor assembly with a lattice structure 10 as presently disclosed with the lattice structure depicted to be suspended out of media and fluid is recirculated through the bioreactor system, according to embodiments of the design.

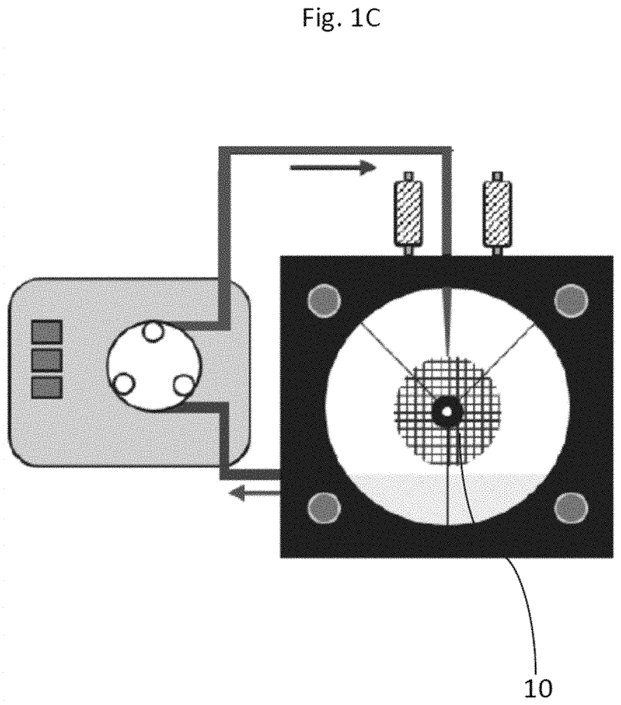

FIG. 2 shows a schematic (left) of a lattice structure according to some embodiments of the design where the lattice structure 10 has a first channel pore surface 20 a and a second channel pore surface 20 b. A photograph (right) of a lattice structure 10 with the plurality of channel pores 15, core passage 25, and sampling ledge members 30 depicted, according to some embodiments of the design.

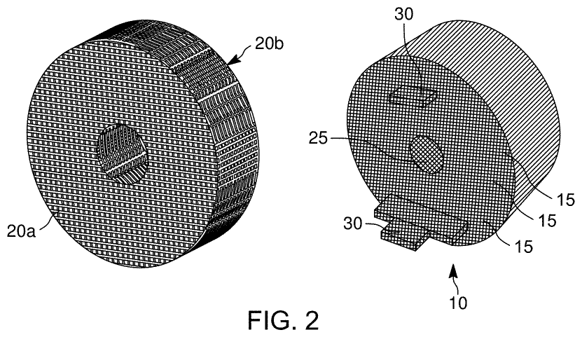

FIG. 3A is a schematic showing sequential (left to right) layer three-dimensional (3D) printing of the lattice structure 10, according to embodiments of the design.

FIG. 3B is a schematic depicting one embodiment of forming the channels and channel pores 15 of the lattice structure 10, according to embodiments of the design.

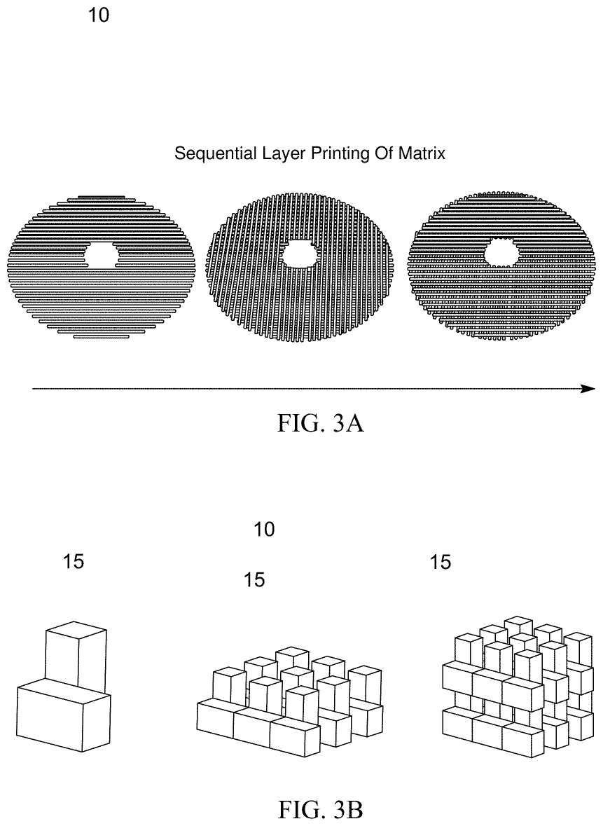

FIGS. 4A, 4B, 4C, 4D, 4E, and 4F are each scanning electron microscope (SEM) images of one or a few channel pores in a polylactic acid (PLA) 3D printed lattice structure where FIG. 4A is a view of an exterior side of the lattice structure 10. FIG. 4B is a view of an interior section of the lattice structure 10. FIG. 4C is a top-down view of the lattice structure 10 with the channel pores 15. FIG. 4D is a zoomed in image of the exterior side of FIG. 4A of the lattice structure 10. FIG. 4E is a zoomed in image of the interior section of FIG. 4B of the lattice structure 10, and FIG. 4F is a zoomed in top-down view of FIG. 4C of the lattice structure 10. The white squares denote a magnified view of that area seen in the second row below the top row.

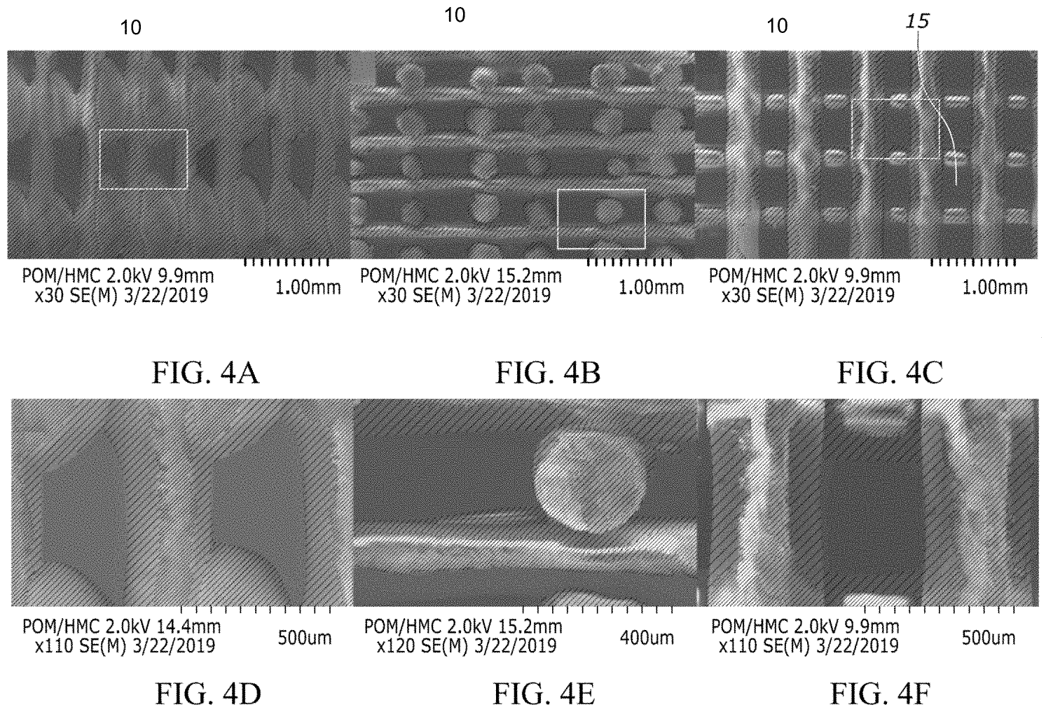

FIG. 5 shows the results from Computational Fluid Dynamic (CFD) modeling on a modeled lattice structure where the upper model is a velocity contour showing the calculated results of fluid velocity in the lattice structure and the lower model shows the calculated results of shear stress in the lattice structure, according to embodiments of the design.

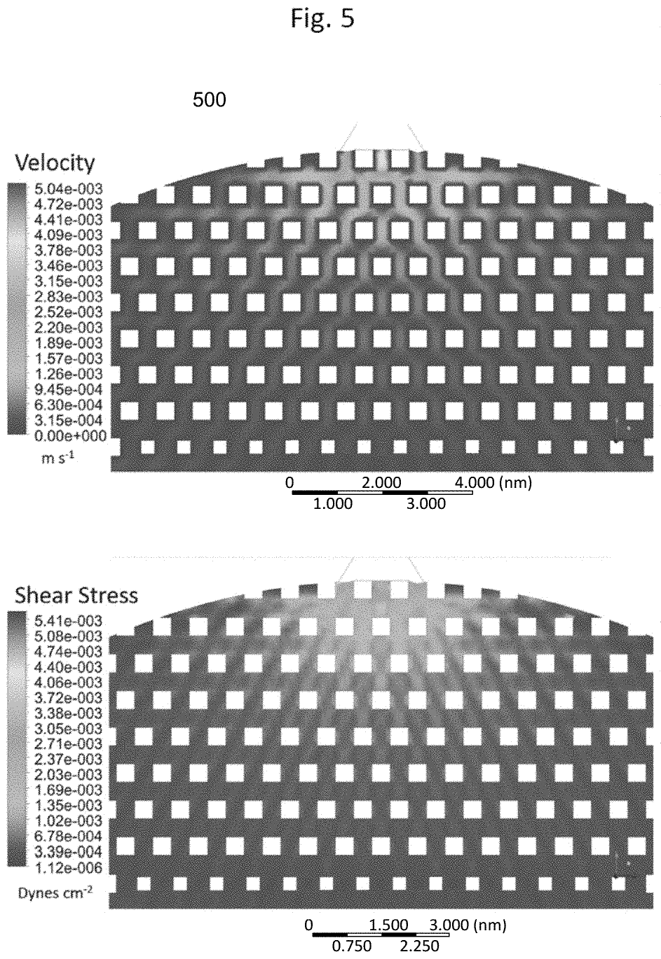

FIG. 6 is a graph 600 of inlet flow rate versus average wall shear where the lattice structure was modeled in ANSYS and tested at various flow rates using Fluent. The strain rate was converted into shear stress using Equation 1, according to embodiments of the design.

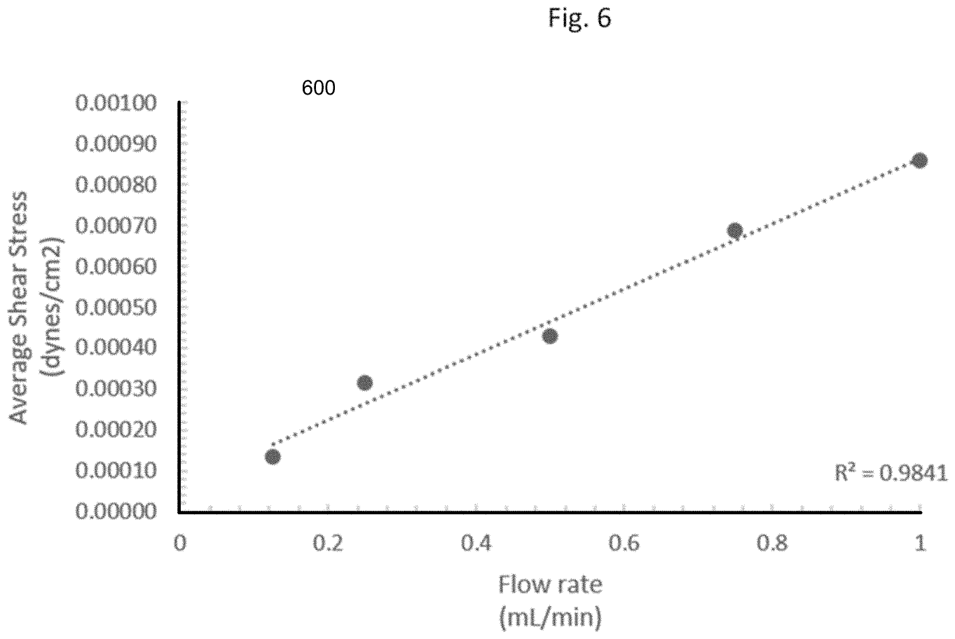

FIG. 7 shows human mesenchymal stem cells (hMSCs) 700 imaged on a PLA lattice structure used in a bioreactor. The hMSCs cells underwent a 3 day prime at 1.5% oxygen and were then cultured out for 7 day, and then stained with phalloidin (red) and DRAQ5 (blue). The two upper images show hMSC on a single fiber. The lower left image is a low magnification showing hMSC coverage among parallel fibers. The lower right image is projected Z-stack of fibers showing cell coverage. Center image shows top view (XY projection), and the top and side bars in this image show sideways projection (ZX and ZY).

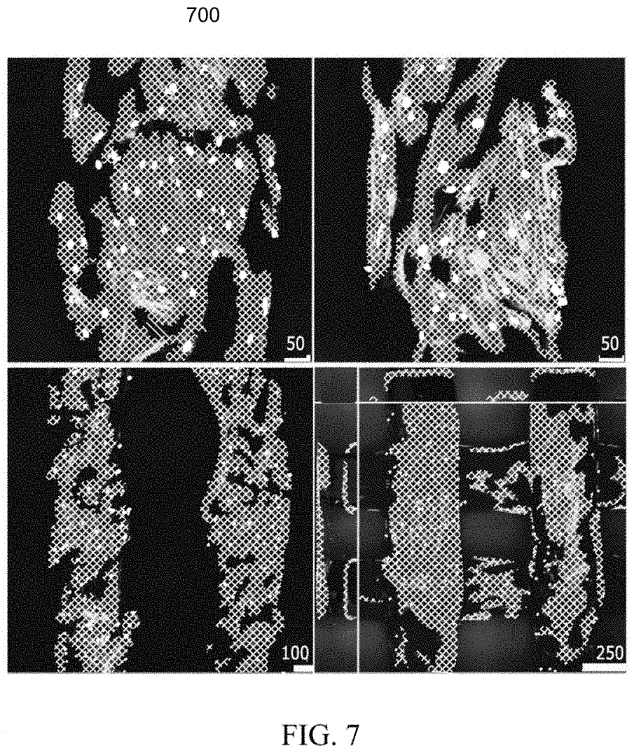

FIGS. 8A-8D are data graphs 800 from a hMSC bioreactor culture of varying oxygen tension compared to static hMSC culture grown under the same oxygen amounts and harvested on day 7. Static refers to t75 tissue culture flasks and dynamic refers to bioreactor culture. FIG. 8A shows the results of cell growth rate (i.e., doubling time) on the bioreactor and static hMSCs with respect to the oxygen tension. FIG. 8B is a graph of the fold increase in the culture growth rate for the hMSC bioreactor and static cultures of FIG. 8A. FIG. 8C is a graph of cells per cm². FIG. 8D is a graph of the specific growth rates and doubling times of hMSC cells grown on a lattice structure in a bioreactor according to embodiments of the design and hMSC cells grown in static culture.

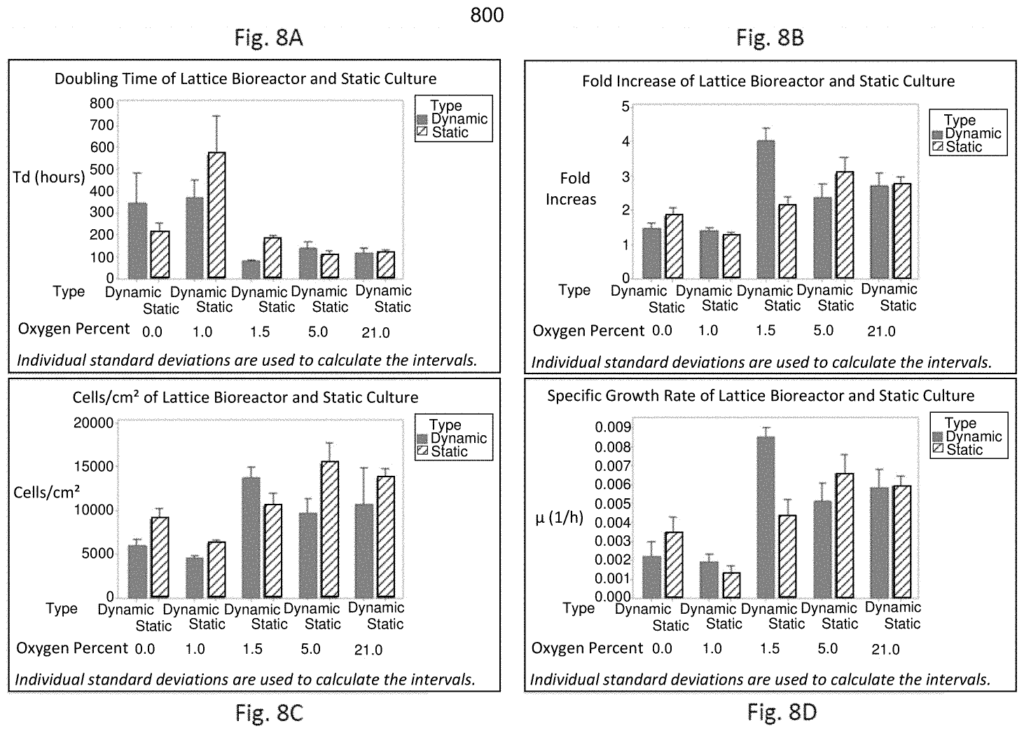

FIG. 9 is an exemplary graph of data 900 obtained from flow cytometry of hMSC grown on a lattice structure in a bioreactor system according to embodiments of the design in which hMSC cells were harvested day 7 from varying oxygen tension. CD105, CD73, CD19 and CD14 stained cells were analyzed using flow cytometry. No significant differences were found in marker expression after preconditioning using 0%, 1%, 1.5%, 5%, and normoxic gasses.

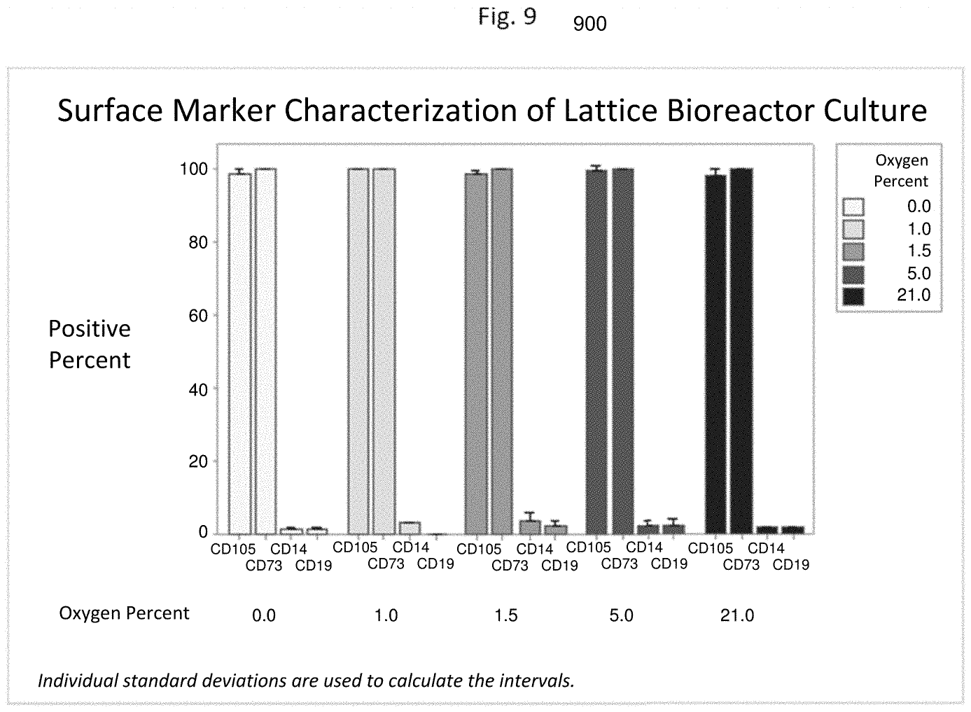

FIG. 10A is an exemplary graph 1000 of hMSC biomarker characterization using flow cytometry. The hMSC cells were cultured in both static and bioreactor (with lattice structure) and compared using CD105, C73, CD19, and CD14 staining, according to embodiments of the design.

FIG. 10b shows exemplary data of an overlaid flow cytometry image 1010 of 1.5% O2 primed hMSC cultures from day 7 in dynamic bioreactors (with lattice structure) showing positive markers and negative markers, according to embodiments of the design.

FIG. 10C shows exemplary data of an overlaid flow cytometry image 1020 of 1.5% O2 primed normoxic polystyrene static control culture showing positive markers and negative markers, according to embodiments of the design.

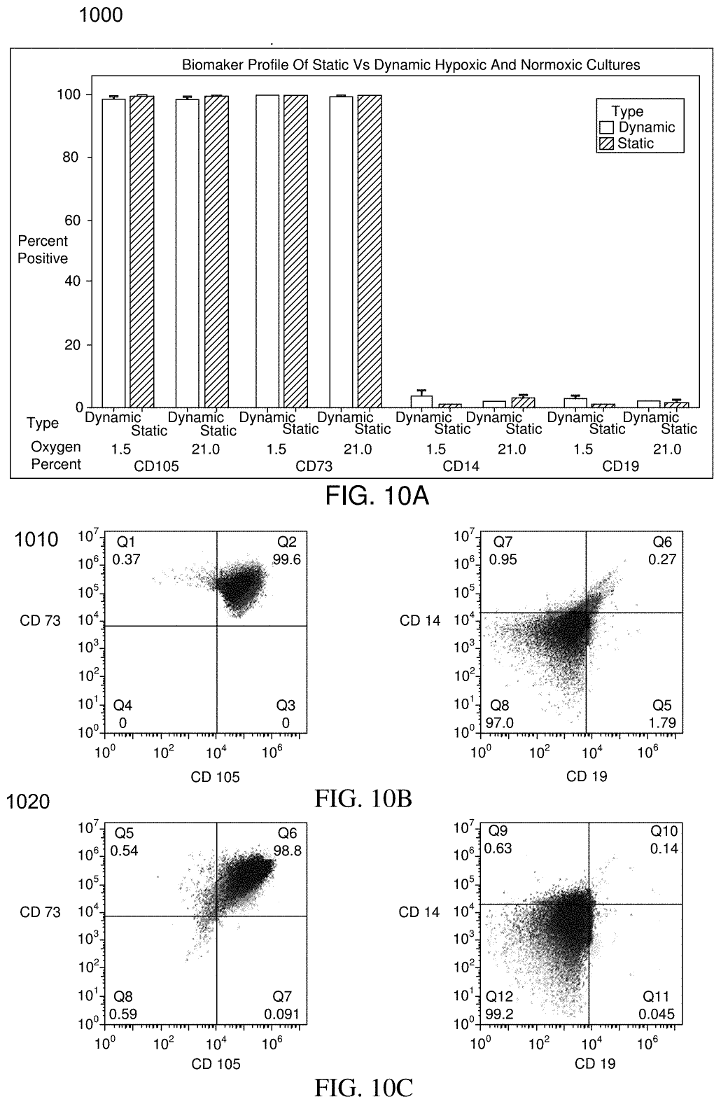

FIG. 11 shows exemplary light microscope images 1100 of stem cell induction in which cells were harvested day 7 from bioreactor (with lattice structure) and cultured in respective differentiation media following specialized protocols. The top row of images show osteocyte cell induction, the middle row shows adipocyte cell induction, and the bottom row shows chondrocyte cell induction. After the allotted time, cells were fixed, stained, and imaged using light microscopy. Scale bars are 100 microns. First column of images are stain controls of respective cultures.

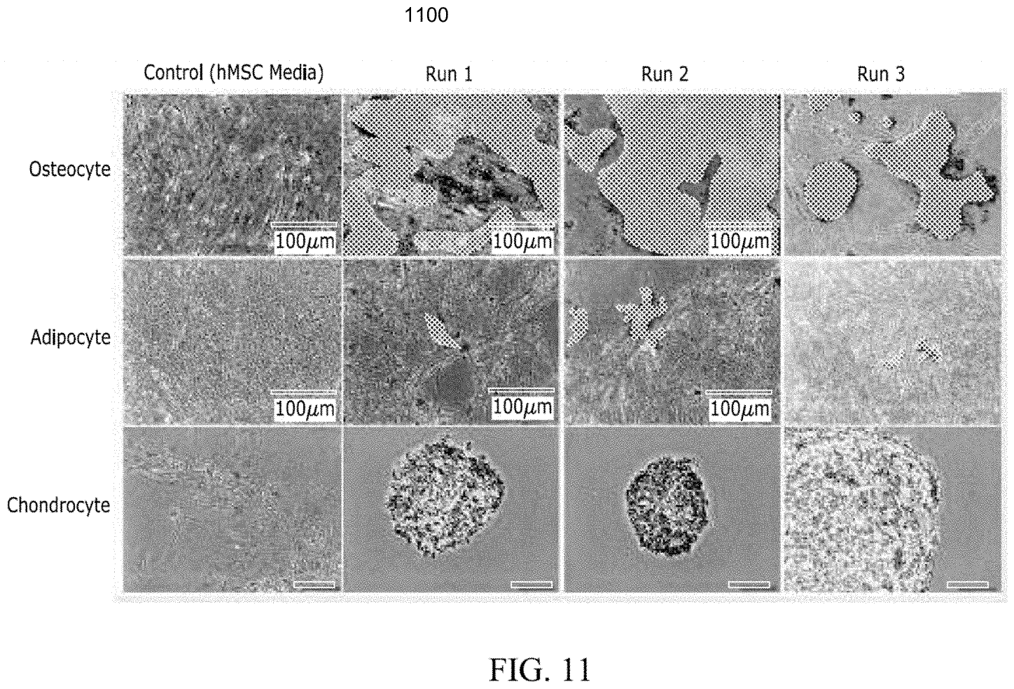

FIGS. 12A and 12B show exemplary bar graph 1200 data comparing culture methods including dynamic bioreactor (with PLA lattice structure), spinner flasks, and static growth in t75 flasks as indicated, in which FIG. 12A presents doubling time and FIG. 12B presents the specific growth rates of the corresponding hMSCs. Static cultures were grown in t75 flasks according to ATCC guidelines. Spinner cultures used Cytodex-1 microcarriers in spinner flask. Dynamic culturing including use of a PLA lattice structure used PLA lattice as per method.

The design includes a lattice structure for culturing cells with high density yields while maintaining cell type homogeneity. The lattice structure is porous and made from a rigid biocompatible material. The porous and rigid lattice design provides for increased surface area and effective media and oxygen exposure under low shear conditions (e.g., low flow rate). Accordingly, the advantageous design enables robust growth of cells yielding high density cultures with high cell purity—i.e., cell type homogeneity.

With reference to FIGS. 1A-1C, the contemplated features of the lattice structure 10 allows for its use in a bioreactor culturing assembly 5. More specifically, with reference to FIG. 2, the lattice structure 10 is made from a biocompatible rigid material and includes a plurality of channels 15 extending between a first channel pore surface 20 a and a second channel pore surface 20 b and each of the plurality of channels having a first channel pore and a second channel pore altogether forming a plurality of channel pores on each of the first channel pore surface and the second channel pore surface. Typically, each of the channel pores has an area of between about 0.01 mm² to about 1 mm², and the biocompatible rigid substrate material having a rigidity as measured by the Young's modulus value of the material, in which the rigidity is at least 0.5 gigapascals (GPa).

In some embodiments, the plurality of channels 15 in the lattice structure 10 are uniform in size and distribution throughout the structure to allow for even distribution of media received by gravity flow. Although any suitable manufacturing method and material may be used, the uniformity of the channels also allows for facile modular fabrication. Suitable manufacturing methods include casting or molding, as well as three-dimensional (3D) printing, computer numerical control (CNC) machining (e.g., CNC milling), selective laser sintering (SLS) (e.g., SLS 3D printing), injection molding, and photolithography. With reference to FIGS. 3A and 3B, fabrication of lattice structure is suitable for 3D printing in which the channels making up the lattice structure can be sequentially layered (FIGS. 3A-3B) allowing for facile fabrication and the advantageous uniformity and distribution of the channels and pores throughout the lattice structure. In an exemplary method using 3D printing, the lattice design is cut into many XY layers using computer software, known as slicing software. Then the printer type heats a material substrate to melting and extrudes the material though a nozzle, creating a very fine filament to complete a full layer. The extrusion head will then increase height in the Z axis and deposit another layer on top of the last. The heat of the filament causes the second layer to fuse to the first and the 3D object is slowly built up by subsequent layer addition.

While a lattice structure having non-uniform or less uniform channels is contemplated, a lattice structure having uniform channels is preferred. The uniformity provides even distribution of media and oxygen to the cells and allows for less turbulence surrounding the structure. Additionally, the manufacturing of a uniform lattice structure allows for easy modular fabrication. While uniformity can be readily achieved using any suitable method, approximate uniformity accounting for minor size variations is contemplated. Variations in channel and pore size and positioning may vary within the lattice structure. As such, uniformity as used herein also includes approximate uniformity and approximately uniform to include variations in any dimension of no more than 2%. Preferably, the variation in any dimension is no more than 1, 0.9, 0.8, 0.7, 0.6, 0.5, 0.4, 0.3, 0.2, or 0.1%.

The contemplated lattice structure may be used as cell culturing platform for any cell type. Any cell type includes animal (e.g., mammalian), yeast, bacterial, and plant cells. Notably, the lattice structure is conducive for growing adherent cells which require anchoring to a surface for growth. In addition to the lattice structure material being biocompatible, the material surface should also have some rigidity for effective adherence of the cells. The inventors contemplate a rigidity greater than that of cellulose and cellulose-based fibers. Typically, the rigidity of the biocompatible substrate material has a Young's modulus value of at least 0.5 gigapascals (GPa). More typically, the rigidity of the biocompatible substrate material is greater than 0.5 GPa. In preferred embodiments, the rigidity of the biocompatible material is at least 0.6, 0.7, 0.8, 0.9, 1.0, 1.1, 1.2, 1.3, 1.4, 1.5 GPa. More preferably, the rigidity of the biocompatible material is at least 1.0 GPa. For example, the rigidity of the biocompatible material may be of between 1.0 GPa to 4.5 GPa. Most preferably, the rigidity of the biocompatible material is of between 1.0 GPa to 4.0 GPa. For example, the rigidity of biocompatible material may be 1.0, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or 4.0 GPa. Any suitably rigid biocompatible material may be used. Exemplary materials for fabrication of the lattice that are suitably rigid and biocompatible include polylactic acid (PLA), polycaprolactone (PCL), acrylonitrile butadiene styrene (ABS), polycarbonate (PC), polystyrene (PS), polyurethane (PU) (e.g, thermoset polyurethane (TPU)), and polyethylene terephthalate glycol (PETG). Selection of a rigid biocompatible material based on the requirements and restrictions for a particular bioreactor or cell culture assembly. Biocompatibility of a material includes sterilization, and any selected material can be sterilized by any suitable sterilization method. For example, heat sterilization at 121° C. (250° F.) or 132° C. (270° F.) (e.g., autoclaving) or by gas or gamma irradiation sterilization. The selected rigid biocompatible material would be selected to have the required rigidity as well as be able to withstand (e.g., without any notable or permanent change to the material) a suitable means of sterilization.

The contemplated lattice structure may be of any suitable size. While the lattice structure as disclosed herein may be used in a bioreactor assembly system, the use of the lattice structure is not limited to a bioreactor assembly system. Accordingly, the lattice structure as disclosed herein may be fabricated to have any size and dimension that is limited only by the capability to manufacture the lattice structure. As specifically exemplified herein, a lattice structure for use in a bioreactor assembly system was fabricated to a size that can be incorporated into the bioreactor assembly system. The skilled person could readily follow this present disclosure to increase the size of the lattice structure, as the disclosed methods and protocols for fabricating a lattice structure of the presently disclosed size and dimensions are applicable for larger as well as smaller structures and would merely be restricted by the limitations of the manufacturing process. For example, the presently disclosed lattice structure may be fabricated by 3D printing. While the methods disclosed herein may be easily calculated to produce a lattice structure on a much larger scale (e.g., 6 inches, 1 foot, or 3 feet in size for at least one dimension), such fabrication may be limited, for example, by access to or the existence of a 3D printer capable of producing a structure of that size. As such, while 3D printing may be a more straightforward and facile manufacturing method, any suitable method including the more laborious casting or molding, as well as CNC machining (e.g., milling), SLS (e.g., SLS 3D printing), injection molding, or photolithography may be used for forming a lattice structure from a rigid biocompatible substrate.

While any size of the lattice structure is contemplated, for use in most bioreactor culturing systems, the lattice structure is sized accordingly. Typically, the lattice structure has a total space-occupying volume of between about 2 cm³ to about 800 cm³. For example, the lattice structure may have a volume of between about 2 cm³ to about 400, 500, 600, 700, or 800 cm³. More typically, the lattice structure has a volume of between about 10 cm³ to about 100, 150, 200, 250, 300, 350, or 400 cm³. Most typically, the lattice structure has a volume of between about 20 cm³ to about 40, 50, 60, 70, 80, 90, or 100 cm³.

The increased surface area provided by the contemplated lattice structure enables high density growth of cells, especially adherent cells. This increased surface area is provided by the channels formed throughout the lattice. With reference again to FIG. 2, at the ends of each channel are the pore openings 15 which altogether form a channel pore surface 20 a, 20 b on each side of the lattice 10. Typically, these channel pore surfaces have a diameter or diagonal of between about 5 mm up to 100 mm. More typically, the diameter or diagonal of the channel pore surface is of between about 10 mm to 40, 50, 60, 70, 80, 90, or 100 mm. Most typically, the diameter or diagonal of the channel pore surface is of between about 20 mm to 60 mm. For example, the diameter or diagonal of the channel pore surface may be 20, 25, 30, 35, 40, 45, 50, 55, or 60 mm.

The layout of the channels in the lattice structure is not limited. For facile and modular fabrication, the channels are contemplated to be formed equidistant apart in parallel rows as shown in FIG. 2. Typically, each of the channel pores are approximately the same size having no more than 2% variation in any dimension. In particular, for increased uniformity and fabrication, the lattice structure may have a distance that is the same for the width and length of the pores as well as the space in each direction between all of the pores, such that the area of the pore is equal to the area of the space between the pores for at least 70% of the channel pore surface. More typically, the area of the pores and the space between the pores is uniform (with no more than 2% variation) for at least 80, 85, or 90% of the channel pore surface.

The overall shape of the lattice structure is not necessarily limited. Exemplary shapes of the channel pore surface that may be more readily manufactured include a circle, an oblong, a square, an octagon, a hexagon, or a rectangle. More preferably, the channel pore surface has a circular or square shape. Additionally, the shape of channels and pores are not limited, but are more easily fabricated in a square or circle having a diameter or diagonal of between about 0.2 mm and 0.4 mm.

Notably, the presently disclosed lattice structure allows for robust cell culturing of cells. In particular, for adherent mammalian cells (e.g., human MSCs), because the lattice structure allows for sufficient surface area for growth and sufficient wetting. Furthermore, the lattice channels allow for low flow rates of media surrounding the lattice structure—e.g., between 0.25 to 0.50 ml/min. As such, more sensitive cells like hMSCs can grow robustly and without turbulence/shear stress that can also induce differentiation.

In particular, as further detailed herein, using Computational Fluid Dynamic (CFD) modeling, very low velocities and subsequently low shear occur inside the lattice structure. Specifically, the computational modeling of this lattice structure reports maximum values of 0.0054 dynes cm⁻². By comparison, CFD of stirred tank reactors utilizing microcarriers report values of approximately 1 to 5 dynes cm⁻². See, Table 1, below. Tubular systems with similar laminar flow patterns report average values of 0.98 dynes cm⁻². These values all fall within 0.02 to 9 dynes cm⁻², a range shown to upregulate osteogenic genes and differentiation in hMSCs. However, the presently disclosed lattice structure was determined to have two orders of magnitude lower than this reference range.

TABLE 1

| | | | | | | | | | | Total | | Shear |
|---|---|---|---|---|---|---|---|---|---|---|---|---|
| | | | | V | SA | | Cell | SC/mL | SC/cm² | SCs | Td | (dynes |
| Name | Type | Classification | Vendor | (mL) | (cm²) | BA:V | type | x10³ | x10⁴ | x10³ | (hr⁻¹) | cm⁻²) |
| — | POMS | Immobilized | — | 110 | 2,800 | 26.5 | hP- | 0.509 | 2.00 | 56 | 30.2 | 8-5 |
| | Matrix | | | | | | MSC | | | | | |
| Quantum | Hollow | Immobilized | TERUMO | 1,440 | 21,000 | 14.6 | hAd- | 0.167 | 1.14 | 240 | 34.1 | 0.3-0.7 |
| | Fiber | | BCT | | | | MSC | | | | | |
| Mobius | STBR | Suspension | M8ipore | 50,000 | 800,000 | 6 | hBM- | 2.00 | 1.68 | 5,000 | 54.0 | 2-40 |
| | | | Sigma | | | | MSC | | | | | |
| Applmex | Wave | Suspension | Applikon | 1,500 | 7,360 | 4.91 | hAd | 0.190 | 3.87 | 285 | 31.2 | 0.1-0.5 |
| | Bug | | | | | | MSC | | | | | |
| Mag 3 | Paddle | Suspension | PBS | 3,000 | — | — | hBM- | 1.90 | — | 5,700 | 63.0 | — |
| | | | | | | | MSC | | | | | |
| Xpansion | Parallel | Immobilized | Pa8 | 1600 | 6,120 | 3.83 | hAd- | 0.181 | 5.4 | 334 | 34.1 | 0.1 |
| Multiplate | Plate | | | | | | MSC | | | | | |
| ICel8s | Random | Immobilized | Pa8 | 1000- | 40,000 | 40 | hBM- | 2.93 | 16 | — | 67.2 | 1-5 |
| | Fiber | | | 5000 | | | MSC | | | | | |
| | Matrix | | | | | | | | | | | |
| In House | Lattice | Immobilized | — | 20 | 122 | 6.3 | hBM- | 0.2 | 2 | 1.8 | 82 | 0.0042 |
| | | | | | | | MSC | | | | | |

Abbreviations:
Volume (V),
SA Surface Area (SA),
Stem Cels (SC),
Doubling Time (Td).

Advantageously, culturing cells on the rigid biocompatible surface of the lattice structure allows for improved and more efficient downstream harvesting and purification compared to other known methods, including culturing with microcarriers (e.g., beads). Microcarrier-based cell culturing of stem cells requires extra steps in order to purify the cells from the beads. For example, some beads require cell detachment using an enzyme (e.g., trypsin), resulting in an extra step of straining the microcarriers from the lifted cells. Furthermore, the byproducts of digestion of these microcarriers is still a concern for final formulation and patient administration. Pursuant to the United States Pharmacopeia (USP) <788> requirements, microcarriers as particulate matter should be removed from injected products. Thus, systems using microcarriers for hMSC therapies require either initial steps or straining and filtration steps to remove microcarriers from cells after dissociation, adding complication and potentially decreasing overall yield through shear. However, cells grown on the presently disclosed lattice structure can be washed in place and lifted with fewer processing steps and without the use of a harsh enzyme like porcine trypsin. For example, cells grown on the presently disclosed lattice structure may be dissociated from the lattice surface using Cell Dissociation Buffer (CDB) (Gibco) and/or a gentle enzyme solution (e.g., TrypLE™, Gibco). Furthermore, it is noted that a lattice structure made with polylactic acid (PLA) is further advantageous because the degradation product of PLA dissolves into solution as lactic acid which can be easily removed through buffer exchange, but it is also biocompatible and broken down in the body naturally.

In additional embodiments, the lattice structure includes a surface coating to enhance cell adhesion of any type of adherent cell. The surface coating may be applied to all or at least some of the available surface area of the lattice structure. In typical embodiments, the surface coating includes at least an application of functional groups to promote cell adhesion. More typically the surface coating includes denatured protein-based fibers, thermoresponsive polymers (i.e., thermopolymers), sodium hydroxide, and/or a plasma treatment.

The selection of a coating surface may depend on the type of cells to be cultured and their degree of sensitivity to the processes required to later detach the cells from the surface coating or treatment. Denatured protein-based fibers include gelatin, collagen type 1, collagen type 2, fibronectin, and/or laminin. For example, denatured collagen adheres to the lattice biocompatible material (e.g., PLA) and also provides a binding motif for the cells, thereby providing a sort of bridge between the lattice surface and the cells. Plasma treatment of the lattice surface exposes hydroxyl groups in the surface material, thereby provide a binding motif for cells. Additionally, thermoresponsive polymers may also be coupled to the surface of the lattice structure. Examples of these temperature responsive polymers include poly(N-isopropylacrylamide) p(NIPAm), poly-(ethylpyrrolidone methacrylate) (pEPM), poly[2-(dimethylamino)ethyl methacrylate] (pDMAEMA), hydroxypropylcellulose, poly(vinylcaprolactame), and polyvinyl methyl ether. Preferably, the thermoresponsive polymer is p(NIPAm) or pEPM. Accordingly, a thermoresponsive polymer may be coated onto the lattice structure followed by the culturing of cells which attach to the polymers on the lattice surface. With a change in temperature, the cells detach from the polymers. See, e.g., Nash et al., 2012, J. Mater. Chem. 22, 19376, the entire content of which is incorporate herein by reference. These cell dissociation processes are less complicated than conventional protocols and are less destructive to the cells, thereby allowing for higher cell yields without the removal of undesirable components (e.g., trypsin or microcarrier beads).

Considered from a different perspective, because of its biocompatibility, cell detachment from the lattice structure, may not be necessary depending on the desired application. For example, as PLA is biocompatible and similar in rigidity to cancellous bone, hMSCs may be expanded and differentiated in-situ. In this way, polymer rigidity may be either avoided or exploited for tailored stem cell differentiation. Harder polymers like PLA, polystyrene, or polycarbonate (PC) may be printed using high temperature 3D printers, and can be easily treated for cell adhesion. Softer, more elastic materials like polyurethane (PU) have been used for stem cell culture and are also readily available materials for 3D printing. Furthermore, culture on more elastic scaffolds, such as alginate encapsulation, may direct hMSCs to differentiate into chondrocytes and has been used in established differentiation protocols. See, e.g., Elsawy et al., 2017, Renew. Sustain. Energy Rev. 79, 1346-1352 and Fromstein et al., 2008, Tissue Eng. Part A, 14, 369-378, the entire contents of both of which are incorporated herein by reference.

As such high density “pure” (homogenous) cells can be effectively cultured using the presently disclosed lattice structure. Methods for maintaining cell type homogeneity (e.g., cell pureness or stemness), include seeding cells in a bioreactor culture system using the lattice structure disclosed herein. As detailed herein, cell type homogeneity may be maintained up to at least 95% up to at least 9 culture passages. Preferably, cell type homogeneity is maintained up to least 97% through at least 9 culture passages. Typically, cell type homogeneity is maintained up to at least 95% through 6 to 9 culture passages. More typically, cell type homogeneity is maintained up to at least 97% through 6 to 9 culture passages. As disclosed in further detail herein, hMSCs grown in a bioreactor using a lattice structure as disclosed herein, produced higher cell density yields compared to other systems, while maintaining cell type homogeneity as measured by expression of stem cell biomarkers. Furthermore, the cells lifted from the lattice structure surface were easily dissociated with open-air steps and required no extra purification. When tested for stemness (i.e., cell potency), the cells readily differentiated into osteocytes and were able to differentiate into adipocytes.

The contemplated lattice structure may also be utilized for the production of secreted product, as cells are adherent to a stationary scaffold. The disclosed lattice structure is ideal for secreted proteins and vesicles. The cells are bound to the lattice structure surface and will release cytokines and exosomes of therapeutic interest into circulating media. Studies of exosomes has shown their usefulness in wound healing and inflammatory diseases. These vesicles are secreted by hMSCs and contain mRNA, cytokines, growth factors, and other signaling molecules involved in healing, and are a major interest for regenerative medicine. The presently disclosed lattice structure may be run in perfusion, allowing simple harvest of the secretome while cells are held stationary in the bioreactor.

The inventors have further contemplated a lattice structure having a removable sampling shelf 25 (FIG. 2) to be attached to or formed on one or both of the channel pore surfaces. The removable sampling shelf allows for a sample of the cell culture to be removed for observation—e.g., to assess culture confluency and health of the cells. The removable sample shelf may be made of any biocompatible rigid material and is preferably made from the same biocompatible rigid material as the lattice structure.

Additionally, the inventors further contemplated a lattice structure adapted to be placed securely in a bioreactor vessel. With reference to FIG. 2, a hollow passage 30 extends between the channel pores surfaces and allows for insertion of a support member through the lattice structure with minimal disruption to the media flow around the lattice structure and the surface area for cell growth.

The following Examples are presented for illustrative purposes only, and do not limit the scope or content of the present application.

### EXAMPLES

### Example 1

A Scaled Bioreactor Culture System. From 3D modeling in Solidworks it was calculated that a 30 mm diameter lattice structure (e.g., as shown in FIG. 2) as disclosed herein, has a theoretical surface area of 225 cm². As a comparison, each 30 mm diameter repeating layer provides 23.5 cm², which equates to a 32-fold increase in surface area when comparing the 3D lattice to the equivalent 2D culture area.

### Example 2

CFD Modeling. To demonstrate the principle of a reactor using the disclosed lattice structure and extrapolate hydrodynamic forces in the lattice, ANSYS FLUENT was used with a simplified model of the growth lattice. SEM images (FIGS. 4A-4F) of the scaffold were taken to understand the printed geometries and properly model the system in FLUENT (FIG. 5). A dye tracer benchtop experiment was used to validate the model. As mentioned, media is cycled to the top of the circular lattice and pulled by gravity through the pores. Because mixing is accomplished through passive means rather than an impeller, the system is inherently very low shear. This was proven by testing a range of flow rates to estimate shear vs flow rate (FIG. 6). All prospective flow rates fell well below 0.4 dynes cm⁻². As 0.25 mL min⁻¹ resulted in the lowest shear while keeping the matrix well wetted, it was the tested flow rate for hMSC culture. At this flow rate CFD modeling reported a maximum of 0.0059 dynes cm⁻² (FIG. 5) and an average of 0.0031 dynes cm⁻² (FIG. 6). The areas of highest shear were at the top and bottom center of the matrix insert, where the media was entering and exiting the lattice respectively.

### Example 3

Spinner flask control. As a comparison, hMSCs growth was also investigated on Cytodex-1 microcarriers in small scale spinner flasks. Static cultures (n=6) showed an average doubling time and specific growth rate of 119.07 hrs±11.23 and 0.0062 hr⁻¹±0.0013 respectively (FIG. 12A). Cells cultured in spinner flasks showed an average doubling time of 113.6 hrs±23.75 and a specific growth rate of 0.0062 hr⁻¹±0.0013 (n=3) (FIG. 12B). Both are significantly longer (p=0.002) compared to lattice reactor (n=5) results.

### Example 4

Cell viability on polylactic acid (PLA) Lattice. Cell viability was compared between culture substrates and static vs dynamic cultures as previous studies with fibrous matrices exhibited increased cytotoxicity. On day 7 of cultures, cells were enzymatically lifted and viability was tested via trypan blue staining. PLA lattices were removed from culture wells to isolate only cells adherent to the PLA lattice. Dynamic PLA cultures from the bioreactor had an average viability of 96.54%±2.82. Cells grown in dynamic bioreactor culture on PLA showed no statistically significant difference (p=0.98) from static PLA culture plates, with an average viability of 96.76%±3.84. Dynamic PLA showed no difference (p=0.45) from Static PS, which had an average viability of 95.13%±1.07. This is also in agreement with the fact that static PLA and PS showed no statistical difference in viability (p=0.38). Therefore, PLA showed no detrimental effects on cell viability in both static and dynamic cultures compared to conventional culture on treated polystyrene flasks.

### Example 5

Dynamic seeding. Because of the larger channel sized and homogeneity of the lattice, a new seeding protocol was developed to increase seeding efficiency. The method that yielded the best results was through static settling of the cells. The volume of media the lattice could hold was found to be 2 mL. Thus, 500,000 cells were resuspended in 2 mL of hMSC media. This cell rich media was then slowly injected through a Luer lock until liquid had cleared the lines. The reactor was then placed into the incubator for 45 minutes to allow cells to settle onto lattice and adhere. Hypoxic gas was then overlaid into the system though the filter ports and the peristaltic pump was then started. After 7 days cells formed confluent monolayers towards the top center of the lattice sampling shelf (FIG. 7).

### Example 6

Reactor Culture. Normoxic reactor culture resulted very similar doubling time as polystyrene (PS) control cultures (FIG. 8A). Because hMSCs normally grow in more comparatively more hypoxic conditions in vivo, oxygen tension was investigated as a means of increasing cell proliferation. It was found that 1.5% O₂ resulted in a four-fold increase in cell yield; double that of conventional flask culture methods tested (p<0.001) (FIG. 8B). Normalized yield to surface area was 13,725 cells cm⁻² at 1.5% O₂ (FIG. 8C). This in-situ conditioning resulted in the significant increase in specific growth rate (0.0085 h⁻¹±0.0005) (FIG. 8D). When lifted and analyzed via flow cytometry it was found that cells cultured in the bioreactor retained their biomarker phenotype regardless of gas composition used for hypoxic treatment (CD105+CD73+CD14− CD19−); ANOVA showed no significant difference in CD105 (p=0.309), CD73 (p=0.347), CD19 (p=0.676), and CD14 (p=0.523) biomarker expression (FIG. 9). Thus, oxygen tension had a drastic effect on cell proliferation, and no effect on biomarker profile. Cultures primed at 0% and 1% (n=3 for both conditions) produced statistically similar cell yields, and cultures primed at 5% and 21% oxygen showed no statistically significant difference via Tukey test at 95% CI. Compared to control cultures on static tissue treated PS, the dynamic bioreactor culture on PLA produced a higher purity MSCs according to ISCT standards, Lifted cells were over 98% dual CD105 and CD73 positive cells in reactor culture compared to 94% in static normoxic polystyrene culture (p=0.005) (FIG. 10A). There was no significant difference in the negative markers CD14 and CD19 under normoxic (n=9) or 1.5% hypoxic conditioning (n=6) (FIG. 10A), and single populations of cells were harvested from bioreactors (FIG. 10B, 10C). Again, cells formed monolayers on the PLA filaments much like control cultures on PS dishes.

### Example 7

Differentiation potential. To test differentiation potential per ISCT guidelines, osteocyte, adipocyte, and chondrocyte inductions were performed stem cells harvested from 7 day bioreactor culture. For inductions, cells were cultured between 15 to 20 days in their respective, defined ATCC differentiation media, after which cells were washed, fixed and stained. After 7 days in bioreactor culture and hypoxic conditioning the cells retained their ability to differentiate into Adipocytes, Chondrocytes, and Osteocytes (FIG. 11). Control cultures were also done in parallel with the inductions and stained with the same dyes. Control cultures showed no staining in uninduced hMSC controls cultured for 21 days in hMSC media.

### Example 8

Stem cell culture. hMSCs were cultured according to guidelines provided from American Type Culture Collection (ATCC). Briefly, cells were cultured in hMSC media (ATCC PCS-500-030) supplemented with the bone marrow derived hMSC bullet kit (ATCC PCS-500-041) at 37° C. and 5% CO₂ on T-75 treated tissue culture flasks. A ¾ media exchange was performed on day 3, and cells were passaged at 80% confluency, usually on days 6 or 7. Cells were lifted using 3.5 mL of 0.25% trypsin and 0.53 mM EDTA solution (ATCC 30-2101) for regular passaging of T-75 flasks, and cells were re-plated at 5,000 cells cm⁻². Cell pelleting was performed by centrifugation at 270×g for 5 minutes. Working cell bank was created from passage 4 hMSCs and stored in liquid nitrogen. Experiments using hMSCs were conducted on cells between passage 5 and 9. Specific growth rate and doubling time were calculated to compare culture success.

Td=(T2−T1)*ln [symbol] (2)ln [symbol] (q2q1) Equation [symbol] [symbol] 1

Equation 1: Doubling time. Where Td is the doubling time, q₂ is the final cell count, and q₁ is the initial cell seeding quantity.

μ=ln [symbol] (q2q1)t Equation [symbol] [symbol] 2

Equation 2: Specific growth rate. Where μ is the specific growth rate, q₂ is the final cell yield and q₁ is the initial cell seeding quantity, and t is the time between q₂ and q₁.

### Example 9

Oxygen tension studies. To induce low oxygen states, cells were placed in a hypoxia chamber (billups-rothenberg) and gas flushed for 6 minutes with the regulator set at 5 PSI and 10 L min⁻¹. Gas composition varied, but was mixed based on PSI. For reactor cultures the mixed gasses were introduced at 100 mL min⁻¹ for 5 minutes to exchange the head space and oxygen from the media. At first tri-gas mixture including 5% CO2 was used, but hMSCs preferred basic conditions and as such CO₂ was excluded from later bioreactor runs with negligible impact on yield and purity. For both cultures the hypoxic gas was overlaid for 3 days, at which point normoxic gas was reintroduced to the culture. For 0% O₂ treatment period had to be reduced to only 1 day.

### Example 10

Reactor construction. The chamber of the reactor is made of a 9 cm long polycarbonate tube with an ID of 2.25 in and an OD of 2.50 in. Four 316 stainless steel barbed hose adapters are tapped into the top of the polycarbonate, two for media circulation and two for gas exchange through 0.2 μm filters. Size 14 silicone hose was used for main liquid handing loop, with a 14 gauge Tygon Pharmed section for peristaltic pumping. The headplate and backplate are made of 316 Stainless steel. The interior reactor components include the 3D printed matrix, which is suspended out of the media using two brackets. Media pumped to the top of the lattice is perfused through the lattice design via gravity, providing gas exchange and nutrients to the cells. Gas control is highly tunable, as there is less liquid for gas to diffuse through to be available to the cells. The front plate of steel has a pass-through port for access to removable sampling scaffolds to monitor cell confluency. Both the lattice matrix and the holding parts were printed from PLA. Minimum and maximum working volumes used were 20 and 30 mL.

### Example 11

Lattice design and bioreactor culture. PLA matrices were 3D printed using a PrintrBot Simple printer and Cura 3D (V3.2.1) printing software. A 0.4 mm nozzle diameter was used to print the PLA scaffold. The lattice is constructed such that the smallest features are printable with a conventional 3D printer, and allow ample space for cells to culture into monolayers. For this extruder the lower limit of resolution was 400 microns in the XY plane. The lumen between fibers was made to be the same width as the fiber itself. Also included into the design are two inserts for non-destructive means of visualization of cell confluence and viability via calcein staining. To sterilize parts before culture, the matrix and supports are assembled and placed into reactor and steam sterilized at 121° C. for 15 minutes under a dry cycle. After sterilization the matrix is washed and wetted with filtered and autoclave-sterilized 1×PBS (VWR VE404) and gelatinized with filtered and autoclave-sterilized 0.1% (WN) gelatin (Fisher 9000-70-8) in MQ water for 45 minutes at 37° C., or overnight at 4° C. The matrix is then rinsed with PBS to remove excess gelatin, and cells are seeded at 2,500 cells cm⁻². Approximate surface area was calculated using Solidworks (Waltham, Mass.) analysis function. To allow cells to adhere only in the lattice the desired number of cells were resuspended first in a total of 2 mL, as this volume was found to be the holding volume of the matrix. Cells were allowed to settle in the matrix for 45 minutes before starting the recirculation loop. Recirculation was run between 0.25 mL and 0.5 mL min⁻¹. A range is noted because as the peristaltic tube relaxed during use, the peristaltic pump tended to speed up, slightly increasing the overall rate. This was the allowable flow rate range because it was the slowest rate that still allowed complete matrix wetting. A % media exchange was performed on day 3, and cells were harvested on day 7 using a lifting cocktail comprised of a 2:1 mixture of Cell Dissociation Buffer (CDB) (Gibco 13151014) and TrypLE-Express (Gibco 12604021). Lifting was accomplished by aspirating media out and cycling 10 mL of PBS through system at 1 ml min⁻¹ to remove residual media. PBS was then aspirated, and cell-lifting cocktail was added. The reactor was then cycled at 0.25 ml min⁻¹ for 30 minutes, or later at 1.5 mL min⁻¹ for 15 minutes. Viability and cell counting was performed using hemocytometer and trypan blue staining.

### Example 12

Microcarrier culture in spinner flask. Cytodex-1 microcarriers were weighed and autoclaved at 121° C. for 15 minutes. Microcarriers were then hydrated in hMSC media. Cells were seeded at 5,000 cells cm⁻² in 50 mL of media in a 250 mL spinner flask (Wheaton). For the first 24 hours, the spinner flask was set to 15 RPM to allow hMSCs time to adhere to the microcarriers, after which agitation was increased to 30 RPM and volume increased to 80 mL. A ½ media change was performed on day 3. Samples were drawn each day and fixed in 4% paraformaldehyde (PFA) for 15 minutes. Cells were then prepared for cell counting via DRAQ5 (Abcam ab108410), staining in a 5 mMol solution overnight. Samples were then washed twice with PBS, allowing microcarriers to gravity settle between washes. Samples were imaged on Leica SP5. The culture was run for a total of 7 days. On day 7 media containing microcarriers was split into 50 falcon tubes and microcarriers allowed to settle for 20 minutes. Media was aspirated and microcarriers washed twice with PBS. When settled again, TrypIE was added and mixture was put back into the incubator for one hour to lift cells for counting and characterization.

### Example 13

Confocal microscopy. Cells were cultured on lattice matrices in the bioreactor for 7 days. Cells were washed using Ca⁺⁺ and Mg⁺⁺ PBS and fixed in place with 4% PFA for 15 minutes and washed again with PBS. Permeabilization was performed using 1% (WN) Triton-X 100 in PBS for 30 minutes at 37° C. Cells were then washed and placed in 1% (WN) Bovine Serum Albumin (BSA) and 0.1% (WN) Triton-X 100 in PBS for one hour at room temperature. Cells were then stained for 30 minutes with 1 drop mL⁻¹ Phalloidin green (Invitrogen) and 1 μl mL⁻¹ DRAQ 5 resulting in a 5 mMol solution in the blocking solution. Cells were washed with Ca⁺⁺ and Mg⁺⁺ PBS and imaged on a Leica SP5 confocal microscope.

### Example 14

SEM imaging. PLA matrices were washed and prepared for electron microscopy. Samples were stuck to 0.5 in slotted stages (TED PELLA 16111) using conductive double-sided copper tape. Samples were imaged at 2 kV using Hitachi SU-70 scanning electron microscope.

### Example 15

Flow cytometry. hMSCs were cultured in experimental conditions and lifted with a 2:1 mixture of CDB and TrypLE-Express lifting cocktail to preserve cell surface markers. Cells were washed in PBS and placed in lifting cocktail for 15 minutes. After neutralization with fresh media, cells were fixed in 4% PFA for 15 minutes, washed twice with PBS, and blocked for 1 hour at room temperature. Blocking solution consisted of 1% (WN) BSA and 0.1% (WN) Triton-X 100 in PBS. Cells were stained for the positive markers CD105 (Invitrogen MHCD10520) and CD73 (Abcam ab157335) and were negative markers CD14 (Abcam ab91146) and CD19 (Abcam ab25510) at 1 μL per 500,000 cells in 500 μL following recommendations. Samples were then run at medium speed (35 μl/min) on a BD Accuri C6 flow cytometer and analyzed using FlowJo (Ashland, Oreg.). Unstained controls were used to gate cells. Fluorophore compensation was done through FlowJo and Fluorescence minus one (FMO) techniques.

### Example 16

hMSC Differentiation and staining. For both adipocyte and osteocyte differentiation, hMSCs were seeded at 12,000 cells cm² and cultured for 3 days in hMSC media following ATCC Toolkit protocols. ATCC differentiation toolkits for Osteocyte (PCS-500-052) and Adipocyte (PCS-500-050) differentiation were used. On the third day media was completely exchanged. For Adipocyte differentiation a conditioning pre-differentiation media was used, and every third day a ½ media change was performed with Adipocyte maintenance media. Osteocyte differentiation did not require a conditioning media and was maintained with only osteocyte toolkit media. On day 20, cells were washed with calcium magnesium free PBS and fixed by 4% PFA at room temperature for 15 minutes. Cells were washed and stained following respective protocols explained below. In brief, cells were washed with MQ water, and visualized on a phase contrast Olympus IX microscope.

### Example 17

Chondrocyte induction was performed according to a combination of ATCC protocols and previous research. Briefly, hMSCs were lifted from reactor using lifting cocktail, and counted. Cells were resuspended in chondrocyte differentiation toolkit (ATCC PCS-500-051) at 1.25×10⁶ cells mL⁻¹. 200 μl of cell laden media was put into 15 ml polypropylene falcon tubes and centrifuged at 270×g for 5 minutes and placed into incubator without resuspending cell pellet. When placed into incubator the tops of the tubes were loosened to allow gas exchange. After 24 hours the pellet was gently suspended via pipetting. Media was changed every 3 days for 21 days total. On day 21 cell aggregates were sliced into 8 μm thick samples using a HM 500 cryostat (Microm) and OTC compound (Tissue Tek 4583) and place onto glass slides. Samples were then stained and visualized on a phase contrast Olympus IX microscope.

Oil Red O was used to stain adipogenic differentiation of hMSCs. A working solution was prepared by mixing 3 ml of Oil Red solution (#0-1391, Sigma) 2 ml of MQ water immediately before. Cells were covered with oil red working solution and stained for 30 minutes at room temperature. Cells were washed twice with MQ water and visualized.

Alizarin Red stain was used to stain osteogenic differentiation of hMSCs. It arrived in working concentration at the proper pH, so no extra formulation was necessary. After fixation cells were washed twice with MQ water, then Alizarin red staining was overlaid onto the cells and left for 15 minutes. Cells were then washed three times with MQ Water and visualized.

Alcian blue was used to stain for chondrogenic differentiation of hMSCs. After cryostat slicing the samples were washed in Ca⁺⁺ Mg⁺⁺ PBS to preserve attachments while removing OTC compound, and fixed for 15 minutes in 4% PFA. The slides were then washed gently in DI water and alcian blue stain was overlaid onto the samples for 30 minutes. After 30 minutes the slides were rinsed with DI water, and then washed with 3% (V/V) glacial acetic acid solution in MQ water to remove excess dye. The cells were then gently rinsed again with DI water and visualized.

### Example 18

Computational fluid dynamic modeling. A simplified model was created in ANSYS 8.1 using a multiphase Volume of Fluid model in ANSYS FLUENT 18.2 (ANSYS Inc., Canonsburg, Pa.). The model consists of an inlet, the lattice made of crossing 0.4 mm square flow channels, a center void where the lattice would be anchored to its support in the system, and an outlet. Viscosity was modeled using Naiver-stokes equations and simulated using Standard K epsilon. Both energy and species transport were included. The SIMPLE pressure-velocity coupling scheme was used to run a transient model. Momentum convergence was set to 10⁻⁸. The inlet velocity was calculated by taking volumetric flow and dividing it by the diameter of the simulated inlet to give velocity. The model was validated comparing velocity in the model to dye experiments. Shear stress was calculated by Equation 3 using reported strain rate.

τ=ηγ Equation 3

Equation 3: Where τ is the shear stress, γ is the strain rate (s⁻¹), and n is the viscosity of the liquid. This was used in conjunction with the lowest flow rate needed to keep the lattice wetted.

### Example 19

Statistics. Graphs and statistics were done using Minitab 17 (Minitab Inc., PA). Error bars on graphs show 2 standard errors. Student's two-tailed t-test was used to determine significance for two data sets. Significance of multiple data sets was performed via one-way ANOVA and Tukey test.

### Additional Examples

The bioreactor can use a number of different culture growing structures for culturing cells including a lattice structure and a support matrix structure. The culture growing structure can include a surface coating, the surface coating selected from the group consisting of functional groups, denatured protein-based fibers, thermopolymers, plasma treatment, and/or sodium hydroxide. The culture growing structure can have thermopolymers that are selected from poly(N-isopropylacrylamide) p(NIPAm), poly-(ethylpyrrolidone methacrylate) (pEPM), poly[2-(dimethylamino)ethyl methacrylate] (pDMAEMA), hydroxypropylcellulose, poly(vinylcaprolactame), or polyvinyl methyl ether.

The cell culture bioreactor device can have a culture chamber for biological cell growth, an external culture medium reservoir operationally connected to said culture chamber, a support matrix having a top end and at least one lower end mounted within said culture chamber, manifold mechanism in fluid communication with said culture medium reservoir for receiving and distributing culture medium, and supported directly above the top end of the support matrix, fluid circulation mechanism for moving culture medium from the reservoir through said support matrix, and aerating mechanism for introducing air into and removing air from said culture chamber. The culture chamber can further include said support matrix mounted within the culture chamber in fluid communication with the manifold mechanism; and at least a portion of at least one lower end of said support matrix contacting said culture medium at the bottom of said chamber The said culture medium flows from said manifold mechanism in a thin film over substantially the entire surface of said support matrix contacting said biological cells with nutrients contained in said culture medium to the bottom of said chamber. The cell culture bioreactor device has said culture medium flows through the support matrix by a gravity-assisted capillary, wicking process. The cell culture bioreactor device has a support matrix that is a sheet having a middle portion, a first end, and a second end. The support matrix has an elongated support rod mounted within the culture chamber directly below the manifold mechanism. The middle portion of the support matrix is draped over the support rod for receiving culture medium from the manifold mechanism and the first end and the second end resting in the culture medium at the bottom of said chamber. The support matrix can be substantially cylindrical-shaped having a first end and a second end, and said manifold mechanism is a receptacle mounted at the top end of said chamber. The first end of the support matrix is mounted to the receptacle for receiving culture medium from the reservoir and the second end is resting in the culture medium at the bottom end of said chamber. The cell culture bioreactor device can have a manifold mechanism that is an elongated, tubular device having a plurality of apertures positioned directly over the support rod, the device receiving culture medium from the fluid circulation mechanism and distributing the culture medium in a substantially vertical direction to the middle portion of the support matrix through the apertures. The cell culture bioreactor device can have a manifold mechanism that is a cylindrical device, the device receiving culture medium from the fluid circulation mechanism and distributing the culture medium in a substantially horizontal direction around the receptacle to the first end of the support matrix through overflow. The support matrix can divide the culture chamber into a first region and at least one second region. The aerating mechanism can comprise: a gas inlet in communication with the first region and a gas outlet in communication with the at least one second region, the gas inlet and gas outlet being operationally connected to a regulated source of atmosphere for the culture chamber that provides a flow of atmosphere from the gas inlet through the support matrix to the gas outlet. The first region of the culture chamber has a first pressure and the at least one second region of the culture chamber has a second pressure, and wherein the first pressure is substantially equivalent to the second pressure. The support matrix is an elastic porous material having continuous open pores formed of interlacing and interconnected fibers and having a hydrophilic surface suitable as a substrate for biological cells, the continuous open pores of permitting substantially equivalent communication with the interior of the culture chamber from any location on the surface of the support matrix. The thin film of the culture medium has a thickness less than approximately one (1 mm) millimeter. The fluid circulation mechanism has a fluid delivery rate for non-turbulently delivering culture medium through the manifold mechanism to the support matrix in a non-turbulent and non-foam causing tribulation. The fluid delivery rate of the fluid circulation mechanism has a value such that there is substantially no back pressure produced by the culture medium flooding the support matrix, thereby obstructing the flow of atmosphere. The cell culture bioreactor device can have a regeneration mechanism. The regeneration mechanism operationally associated with the fluid circulation mechanism, the regeneration mechanism (a) receiving the culture medium from the outlet, (b) optionally removing waste material or extracting product from such culture medium, (c) optionally replenishing nutrients to the culture medium, and (d) delivering the culture medium to the fluid circulation mechanism.

A method of culturing biological cells can be used to provide a culture chamber for cell growth. The method includes steps (but does not need all of the steps) as follows:

i. positioning a culture medium receptacle above the culture chamber; introducing air into and removing air from the culture chamber;

ii. mounting a support matrix within the culture chamber with at least a portion of the support matrix contacting the culture medium at the bottom end of the chamber;

iii. moving culture medium from the reservoir into the culture chamber; receiving the culture medium into the culture chamber;

iv. distributing culture medium over the support matrix; and

v. allowing the growth of biological cells on the support matrix within the culture chamber.

vi. distributing the culture medium in a thin film over substantially the entire surface of the support matrix and into the culture medium reservoir.

vii distributing the culture medium through the support matrix by a gravity-assisted capillary wicking process.

viii. dividing the culture chamber into a first region and at least one second region;

ix. providing a gas inlet in communication with the first region;

x. providing a gas outlet in communication with the at least one second region; and operationally connecting the gas inlet and gas outlet to a regulated source of atmosphere for the culture chamber that provides a flow of atmosphere from the gas inlet through the support matrix to the gas outlet.

xi. providing a fluid delivery rate for non-turbulently delivering culture medium through the support matrix in a non-turbulent and non-foam causing tribulation.

xii. receiving the culture medium from the outlet and 1) optionally removing waste material or extracting product from such culture medium and 2) optionally replenishing nutrients to the culture medium; and delivering the culture medium to the fluid circulation mechanism.

The support matrix is a sheet having a middle portion, a first end, and a second end. The support matrix: i) mounts an elongated support rod within the culture chamber; ii) drapes the middle portion of the support matrix over the support rod; resting the first end and the second end in the culture medium at the bottom of said chamber; and iii) distributes the culture medium over the middle portion of the support matrix such that it flows evenly through both sides of said support matrix into the bottom end of said chamber. The support matrix can be substantially cylindrical-shaped having a first end and a second end. The support matrix mounts a receptacle to the manifold mechanism; secures the first end of the support matrix to the receptacle; resting the second end in the culture medium; and contacts said first end of the support matrix with the culture medium.

The first region of the culture chamber can have a first pressure and the at least one second region of the culture chamber has a second pressure, and wherein the first pressure is substantially equivalent to the second pressure.

The support matrix can be an elastic porous material having continuous open pores formed of interlacing and interconnected fibers and having a hydrophilic surface suitable as a substrate for biological cells, the continuous open pores of permitting substantially equivalent communication with the interior of the culture chamber from any location on the surface of the support matrix. The thin film of the culture medium has a thickness less than approximately one (1 mm) millimeter.

The fluid delivery rate of the fluid circulation mechanism can have a value such that there is substantially no back pressure produced by the culture medium flooding the support matrix, thereby obstructing the flow of gases.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

Unless the context dictates the contrary, all ranges set forth herein should be interpreted as being inclusive of their endpoints and open-ended ranges should be interpreted to include only commercially practical values. Similarly, all lists of values should be considered as inclusive of intermediate values unless the context indicates the contrary.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

The recitation of ranges of values herein is merely intended to serve as a shorthand method of referring individually to each separate value falling within the range. Unless otherwise indicated herein, each individual value with a range is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the design and does not pose a limitation on the scope of the invention otherwise claimed. No language in the specification should be construed as indicating any non-claimed element essential to the practice of the design.

Groupings of alternative elements or embodiments of the design disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification refers to at least one of something selected from the group consisting of A, B, C . . . and N, the text should be interpreted as requiring only one element from the group, not A plus N, or B plus N, etc.

The foregoing exemplary descriptions and the illustrative preferred embodiments of the present design have been explained in the drawings and described in detail, with varying modifications and alternative embodiments being taught. While the preferred embodiments of the subject design, as described herein, have been presented for purposes of illustration and description and for a better understanding of the invention, this discussion is not intended to be exhaustive or to limit the invention to the precise form disclosed; and obviously many modifications and variations are possible in light of the above teaching. The particular embodiments were chosen and described in some detail to best explain the principles of the design and its practical application to thereby enable others skilled in the relevant art to best utilize the design in various embodiments and with various modification as are suited to the particular use contemplated. It is intended that the invention be defined by the claims appended hereto. Furthermore, the invention as disclosed herein, may be suitably practiced in the absence of the specific elements which are disclosed herein.

1. A cell culture bioreactor device, comprising:
a culture chamber for biological cell growth, an external culture medium reservoir operationally connected to said culture chamber, a culture growing structure having a top end and at least one lower end mounted within said culture chamber, manifold mechanism in fluid communication with said culture medium reservoir for receiving and distributing culture medium, and supported directly above the top end of the culture growing structure, fluid circulation mechanism for moving culture medium from the reservoir through said culture growing structure, and aerating mechanism for introducing air into and removing air from said culture chamber, where the culture growing structure is at least one of 1) a support matrix using wicking and 2) a lattice structure, that is coated with a thermal responsive polymer, where a material of interest growing on the culture growing structure is removed by changing a temperature that the thermal responsive polymer on the surface of the culture growing structure is exposed to and release the thermal responsive polymer along with the material of interest from the remainder of the culture growing structure.

2. The culture chamber of claim 1, further comprising:
said support matrix mounted within the culture chamber in fluid communication with the manifold mechanism;
at least a portion of at least one lower end of said support matrix contacting said culture medium at the bottom of said chamber; and
wherein said culture medium flows from said manifold mechanism in a thin film over substantially the entire surface of said support matrix contacting said biological cells with nutrients contained in said culture medium to the bottom of said chamber.

3. The cell culture bioreactor device of claim 1 wherein said culture medium flows through the support matrix by a gravity-assisted capillary, wicking process.

4. The cell culture bioreactor device of claim 1 wherein the support matrix is a sheet having a middle portion, a first end, and a second end, and an elongated support rod mounted within the culture chamber directly below the manifold mechanism; wherein the middle portion of the support matrix is draped over the support rod for receiving culture medium from the manifold mechanism and the first end and the second end resting in the culture medium at the bottom of said chamber.

5. The cell culture bioreactor device of claim 1 wherein the support matrix is substantially cylindrical-shaped having a first end and a second end, and said manifold mechanism is a receptacle mounted at the top end of said chamber, wherein the first end of the support matrix is mounted to the receptacle for receiving culture medium from the reservoir and the second end is resting in the culture medium at the bottom end of said chamber.

6. The cell culture bioreactor device of claim 2 wherein the manifold mechanism is an elongated, tubular device having a plurality of apertures positioned directly over the support rod, the device receiving culture medium from the fluid circulation mechanism and distributing the culture medium in a substantially vertical direction to the middle portion of the support matrix through the apertures.

7. The cell culture bioreactor device of claim 2 wherein the manifold mechanism is a cylindrical device, the device receiving culture medium from the fluid circulation mechanism and distributing the culture medium in a substantially horizontal direction around the receptacle to the first end of the support matrix through overflow.

8. The cell culture bioreactor device of claim 1 wherein the support matrix divides the culture chamber into a first region and at least one second region, the aerating mechanism comprise:
a gas inlet in communication with the first region and a gas outlet in communication with the at least one second region, the gas inlet and gas outlet being operationally connected to a regulated source of atmosphere for the culture chamber that provides a flow of atmosphere from the gas inlet through the support matrix to the gas outlet.

9. The cell culture bioreactor device of claim 8 wherein the first region of the culture chamber has a first pressure and the at least one second region of the culture chamber has a second pressure, and wherein the first pressure is substantially equivalent to the second pressure.
